(19) United States
(12) Patent Application Publication
(10) Pub. No.: US 2010/0087474 A1
(43) Pub. Date: Apr. 8, 2010

(54) MATERIALS AND METHODS FOR ENHANCED DEGRADATION OF MUTANT PROTEINS ASSOCIATED WITH HUMAN DISEASE

(75) Inventors: Shalesh Kaushal, Gainesville, FL (US); Ritu Malhotra, Gainesville, FL (US); William A. Dunn, Gainesville, FL (US)

Correspondence Address: EDWARDS ANGELL PALMER & DODGE LLP, P.O. BOX 55874, BOSTON, MA, 02205, US

(73) Assignee: UNIVERSITY OF FLORIDA, Gainesville, FL (US)

(21) Appl. No.: 11/919,371

(22) PCT Filed: Apr. 27, 2006
(86) PCT No.: PCT/US2006/016368

§ 371 Date: Dec. 9, 2009

Related U.S. Application Data
(60) Provisional application No. 60/675,143, filed on Apr. 27, 2005.
(60) Provisional application No. 60/723,288, filed on Oct. 3, 2005.

(51) Int. Cl. A61K 31/436; G01N 33/53; A61K 9/00; C12N 5/071; A61K 31/415; A61P 27/02

(52) U.S. Cl. 514/291; 435/7.21; 424/427; 435/325; 514/398

(57) ABSTRACT

The invention features compositions and methods that are useful for treating or preventing a protein conformation disease in a subject by enhancing the degradation of misfolded proteins in vivo.

### CROSS-REFERENCE TO RELATED APPLICATIONS

This application claims the benefit of the following U.S. Provisional Application Nos.: 60/675,143, which was filed on Apr. 27, 2005, and 60/723,288, which was filed on Oct. 3, 2005; the entire contents of each of these applications is hereby incorporated by reference.

### STATEMENT OF RIGHTS TO INVENTIONS MADE UNDER FEDERALLY SPONSORED RESEARCH

This work was supported by a National Eye Institute Grant, Grant No. EY016070-01. The government may have certain rights in the invention.

### BACKGROUND OF THE INVENTION

Proteins must fold into their correct three-dimensional conformation to achieve their biological function. The native conformation of a polypeptide is encoded within its primary amino acid sequence, and even a single mutation in an amino acid sequence can impair the ability of a protein to achieve its proper conformation. When proteins fail to fold correctly, the biological and clinical effects can be devastating. Protein aggregation and misfolding are primary contributors to many human diseases, such as autosomal dominant retinitis pigmentosa, Alzheimer's disease, α1-antitrypsin deficiency, cystic fibrosis, nephrogenic diabetes insipidus, and prion-mediated infections. In other protein-folding disorders, such as autosomal dominant retinitis pigmentosa, age-related macular degeneration, Alzheimer's disease, Parkinson's disease, and Huntington's disease, pathology results because of the cytotoxic effects of the misfolded protein.

Misfolded proteins are recognized by the ER quality control system and are targeted for degradation by the proteasome. Besides the proteasomal pathway, autophagy is another major cellular mechanism for protein degradation. While autophagy can be stimulated by a variety of intracellular and extracellular stresses including amino-acid starvation, aggregation of misfolded protein, and accumulation of damaged organelles, autophagy appears to be a largely non-selective process. Aggregate prone polyglutamine and polyalanine expanded proteins associated with Huntington's disease are degraded by autophagy, and inhibition of autophagy reduced the toxicity of mutant Huntington proteins in fly and mouse models of Huntington disease. Autophagy has also been shown to contribute to the elimination of proteins accumulated in the ER. If methods for increasing autophagy were available, they might enhance the elimination of misfolded proteins, and eliminate the cytotoxic effects associated with their accumulation. Current methods for alleviating the cytotoxic effects of misfolded proteins and preserving neuronal function are urgently required.

### SUMMARY OF THE INVENTION

The invention features compositions and methods that are useful for treating or preventing a Protein Conformation Disease (PCD) by enhancing the degradation of misfolded proteins.

In one aspect, the invention generally provides a method for treating or preventing a protein conformation disorder (PCD) in a subject, the method involving administering an effective amount of a compound that enhances autophagic protein degradation to the subject (e.g., human patient). In one embodiment, the compound (e.g., rapamycin, farnesyl transferase inhibitor, FTI-277, or analogs thereof) inhibits the mammalian target of rapamycin (mTOR) or inhibits Ras homolog enriched in brain (Rheb). In another embodiment, the PCD is any one or more of the group consisting of α1-antitrypsin deficiency, cystic fibrosis, Huntington's disease, Parkinson's disease, Alzheimer's disease, nephrogenic diabetes insipidus, cancer, and Jacob-Creutzfeld disease. In yet another embodiment, the PCD is an ocular PCD selected from any one or more of retinitis pigmentosa, age-related macular degeneration (e.g., wet or dry), glaucoma, corneal dystrophies, retinoschises, Stargardt's disease, autosomal dominant druzen, and Best's macular dystrophy. In still other embodiments, the method further involves administering 11-cis-retinal, 9-cis-retinal, or a 7-ring locked isomer of 11-cis-retinal to the subject (e.g., human patient).

In another aspect, the invention provides a method for treating or preventing an ocular protein conformation disorder (PCD) in a subject (e.g., human patient), the method involving administering an effective amount of a compound that enhances autophagic protein degradation to the subject.

In another aspect, the invention provides a method for treating or preventing retinitis pigmentosa or macular degeneration in a subject (e.g., human patient), the method involving administering to the subject a compound that enhances autophagic protein degradation; and administering 11-cis-retinal or 9-cis-retinal, where the 11-cis-retinal or 9-cis-retinal and the compound are administered simultaneously or within fourteen days of each other in amounts sufficient to treat or prevent retinitis pigmentosa or macular degeneration in the subject.

In another aspect, the invention provides a method for treating or preventing a protein conformation disorder (PCD), where the PCD is any one or more of α1-antitrypsin deficiency, cystic fibrosis, Huntington's disease, Parkinson's disease, Alzheimer's disease, nephrogenic diabetes insipidus, cancer, and Jacob-Creutzfeld disease, in a subject (e.g., human patient), the method involving administering a compound that enhances autophagy in an amount sufficient to treat or prevent the PCD in the subject. In one embodiment, the invention further involves the step of identifying the patient as having a PCD. In yet another embodiment, the invention further involves the step of measuring the level or expression of a misfolded protein, an autophagic marker or autophagic vacuoles in a cell. In one embodiment, the PCD is cystic fibrosis and the method further involves administering an agent selected from any one or more of antibiotics, vitamins A, D, E, and K supplements, albuterol bronchodilation, dornase, and ibuprofen. In yet another embodiment, the PCD is Huntington's disease and the method further involves administering an agent selected from any one or more of haloperidol, phenothiazine, reserpine, tetrabenazine, amantadine, and co-Enzyme Q10. In yet another embodiment, the PCD is Parkinson's disease and the method further involves administering an agent selected from any one or more of levodopa, amantadine, bromocriptine, pergolide, apomorphine, benserazide, lysuride, mesulergine, lisuride, lergotrile, memantine, metergoline, piribedil, tyramine, tyrosine, phenylalanine, bromocriptine mesylate, pergolide mesylate, antihistamines, antidepressants, and monoamine oxidase inhibitors. In yet another embodiment, the PCD is Alzheimer's disease, and the method further involves administering an agent selected from any one or more of donepezil, rivastigmine, galantamine, and tacrine. In yet another embodiment, the PCD is nephrogenic diabetes insipidus and the method further involves administering an agent selected from any one or more of chlorothiazide/hydrochlorothiazide, amiloride, and indomethacin. In yet another embodiment, the method further involves administering an agent selected from any one or more of abiraterone acetate, altretamine, anhydrovinblastine, auristatin, bexarotene, bicalutamide, BMS184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunin.

In another aspect, the invention provides a method of enhancing the degradation of a misfolded protein in a cell (e.g., an ocular cell, a neuron, an epithelial cell), the method involving contacting a cell with an effective amount of a compound that enhances autophagy. In one embodiment, the method further involves the step of measuring the level or expression of a misfolded protein, an autophagic marker or autophagic vacuoles in a cell (e.g., a mammalian cell, such as a human cell, in vitro or in vivo). In yet another embodiment, the method further involves contacting the ocular cell with 11-cis-retinal, 9-cis-retinal, or a 7-ring locked isomer of 11-cis-retinal. In yet another embodiment, the cell contains a mutant protein (e.g., opsin, myocilin, lipofuscin, β-H3 protein) that forms an aggregate or a fibril. In yet another aspect, the invention provides a pharmaceutical composition for the treatment of a PCD comprising an mTOR inhibitor or an analog thereof in a pharmaceutically acceptable excipient.

In yet another aspect, the invention provides a pharmaceutical composition for the treatment of an ocular PCD comprising an effective amount of a compound that enhances autophagy in a pharmaceutically acceptable excipient.

In yet another aspect, the invention provides a pharmaceutical composition for the treatment of retinitis pigmentosa or age related macular degeneration comprising an effective amount of 11-cis-retinal or 9-cis-retinal and an effective amount of an autophagy inhibitor in a pharmaceutically acceptable excipient.

In yet another aspect, the invention provides a kit for the treatment of an ocular PCD, the kit comprising an effective amount of 11-cis-retinal or 9-cis-retinal and an effective amount of rapamycin or an analog thereof.

In yet another aspect, the invention provides a kit for the treatment of retinitis pigmentosa or age related macular degeneration, the kit comprising an effective amount of 11-cis-retinal or 9-cis-retinal; and an effective amount of rapamycin or an analog thereof.

In yet another aspect, the invention provides a method for identifying a compound useful for treating a subject (e.g., human patient) having a PCD, the method involving contacting a cell in vitro expressing a misfolded protein with a candidate compound; and determining an increase in autophagy in the cell relative to a control cell, where an increase in autophagy in the contacted cell identifies a compound useful for treating a subject having a PCD.

In yet another aspect, the invention provides a method for identifying a compound useful for treating a subject (e.g., human patient) having retinitis pigmentosa or age-related macular degeneration, the method involving contacting a cell expressing a misfolded protein in vitro with 11-cis-retinal (e.g., a 7-ring locked isomer of 11-cis-retinal) or 9-cis-retinal, and a candidate compound; and determining an increase in autophagy in the cell relative to a control cell, where an increase in autophagy in the contacted cell identifies a compound useful for treating a subject having retinitis pigmentosa.

In yet another aspect, the invention provides a method for identifying a compound useful for treating a subject (e.g., human patient) having cystic fibrosis, the method involving contacting a cell in vitro expressing a misfolded protein with a candidate compound; and determining an increase in autophagy in the cell relative to a control cell, where an increase in autophagy in the contacted cell identifies a compound useful for treating a subject having cystic fibrosis. In one embodiment, the misfolded protein contains a mutation. In another embodiment, the misfolded protein is an opsin, such as an opsin that contains a P23H mutation. In yet another embodiment, an increase in autophagy is determined by monitoring the level of a protein; by monitoring the expression of an autophagic marker; or by monitoring the number of autophagic vacuoles.

In yet another aspect, the invention provides a method for treating or preventing a protein conformation disorder (PCD) in a subject (e.g., human patient), the method involving administering an effective amount of a compound that enhances a rapamycin or FTI-277 biological activity. In one embodiment, the compound is administered in combination with rapamycin or an analog thereof or is administered in combination with FTI-277 or an analog thereof. In yet another embodiment, the PCD is selected from any one or more of α1-antitrypsin deficiency, cystic fibrosis, Huntington's disease, Parkinson's disease, Alzheimer's disease, nephrogenic diabetes insipidus, cancer, and Jacob-Creutzfeld disease. In another embodiment, the PCD is an ocular PCD selected from any one or more of retinitis pigmentosa, age-related macular degeneration, glaucoma, corneal dystrophies, retinoschises, Stargardt's disease, autosomal dominant druzen, and Best's macular dystrophy. In another embodiment, the method further involves administering 11-cis-retinal, 9-cis-retinal, or a 7-ring locked isomer of 11-cis-retinal to the subject.

In another aspect, the invention provides a method for treating or preventing retinitis pigmentosa in a subject (e.g., human patient), the method involving administering to the subject rapamycin or FTI-277 and a compound that enhances a rapamycin or FTI-277 biological activity. In one embodiment, the method further involves administering 11-cis-retinal or 9-cis-retinal, where the 11-cis-retinal or 9-cis-retinal and the compound are administered simultaneously or within fourteen days of each other in amounts sufficient to treat or prevent retinitis pigmentosa in the subject.

In yet another aspect, the invention provides a method for treating or preventing a protein conformation disorder (PCD) in a subject (e.g., human patient), the method involving administering rapamycin or FTI-277 or an analog thereof in combination with a compound that enhances a rapamycin or FTI-277 biological activity, where rapamycin or FTI-277 and the compound are each administered in an amount sufficient to treat or prevent the PCD in the subject.

In yet another aspect, the invention provides a method of enhancing the degradation of a misfolded protein in a cell, the method involving contacting a cell (e.g., an ocular cell, neuronal cell, epithelial cell) with an effective amount of rapamycin, FTI-277 or an analog thereof and a compound that enhances a rapamycin or FTI-277 biological activity, where rapamycin or FTI-277 and the compound are each administered in an amount sufficient to enhance degradation of the protein. In one embodiment, the method further involves contacting the cell with 11-cis-retinal, 9-cis-retinal, or a 7-ring locked isomer of 11-cis-retinal.

In yet another aspect, the invention provides a pharmaceutical composition for the treatment of an ocular PCD comprising rapamycin, FTI-277 or an analog thereof and a compound that enhances a rapamycin or FTI-277 biological activity in a pharmaceutically acceptable excipient, where rapamycin or FTI-277 and the compound are each present in an amount sufficient to treat or prevent the PCD in the subject (e.g., human patient).

In yet another aspect, the invention provides a method for identifying a compound useful for treating a subject (e.g., human patient) having a PCD, the method involving contacting a cell in vitro expressing a misfolded protein with a candidate compound in the presence or absence of an autophagy enhancer; and determining an increase in autophagy in the cell relative to a control cell, where an increase in autophagy in the contacted cell identifies a compound useful for treating a subject having a PCD.

In yet another aspect, the invention provides a method for identifying a compound useful for treating a subject (e.g., human patient) having retinitis pigmentosa, the method involving contacting a cell expressing a misfolded opsin protein in vitro with 11-cis-retinal or 9-cis-retinal and rapamycin or FTI-277, and a candidate compound; and determining an increase in autophagy in the cell relative to a control cell, where an increase in autophagy in the contacted cell identifies a compound useful for treating a subject having retinitis pigmentosa.

In yet another aspect, the invention provides a method for identifying a compound useful for treating a subject (e.g., human patient) having cystic fibrosis, the method involving contacting a cell in vitro expressing a misfolded CFTR protein with a candidate compound and rapamycin or FTI-277; and determining an increase in autophagy in the cell relative to a control cell, where an increase in autophagy in the contacted cell identifies a compound useful for treating a subject having cystic fibrosis.

In various embodiments of any of the above aspects, the PCD is selected from any one or more of α1-antitrypsin deficiency, cystic fibrosis, Huntington's disease, Parkinson's disease, Alzheimer's disease, nephrogenic diabetes insipidus, cancer, and Jacob-Creutzfeld disease. In yet other embodiments of any of the above aspects, the PCD is an ocular PCD selected from any one or more of retinitis pigmentosa, age-related macular degeneration (e.g., wet or dry form), glaucoma, corneal dystrophies, retinoschises, Stargardt's disease, autosomal dominant druzen, and Best's macular dystrophy. In still other embodiments of any of the above aspects, the compound inhibits the mammalian target of rapamycin (mTOR) or inhibits Ras homolog enriched in brain (Rheb). In one preferred embodiment of any of the above aspects, the ocular PCD is retinitis pigmentosa or macular degeneration, such as age-related macular degeneration (e.g., the dry or wet form). In still other embodiments of any of the above aspects, the method further comprises administering 11-cis-retinal, 9-cis-retinal, or a 7-ring locked isomer of 11-cis-retinal to the subject (e.g., a mammal, such as a human). In preferred embodiments of any of the above aspects, the subject comprises a mutation that affects protein folding (e.g., a mutation is in an opsin, such as a P23H mutation or a mutation in CFTR, such as ΔF508). In other embodiments of the above aspects, the degradation is selective for the misfolded protein. In still other embodiments of any of the above aspects, a compound useful in a method of the invention is rapamycin, a farnesyl transferase inhibitor, FTI-277, or an analog of such a compound. In still other embodiments, the method further involves administering 11-cis-retinal, 9-cis-retinal, or a 7-ring locked isomer of 11-cis-retinal to the subject. In yet another embodiment, the compound inhibits the mammalian target of rapamycin (mTOR) or inhibits Ras homolog enriched in brain (Rheb). In various embodiments of any of the above aspects, the subject (e.g., a human or veterinary patient) contains a mutation that affects protein folding, such as a mutation in opsin (e.g., a P23H mutation). In still other embodiments, the degradation is selective for the misfolded protein. In still other embodiments, the 11-cis-retinal or 9-cis-retinal and the compound are administered within twenty-four hours, within three days, or within five days of each other. In another embodiment of any of the above aspects, the 11-cis-retinal or 9-cis-retinal and the compound are administered simultaneously. In still other embodiments, the 11-cis-retinal or 9-cis-retinal and the compound are administered to the eye; for example, the administration is intra-ocular. In yet another embodiment, the 11-cis-retinal or 9-cis-retinal and the compound are each incorporated into a composition that provides for their long-term release (e.g., a microsphere, nanosphere, or nanoemulsion). In yet another embodiment, the long-term release is via a drug delivery device. In yet another embodiment, the method further involves administering a vitamin A supplement. In still other embodiments of any of the above aspects, an increase in autophagy is determined by monitoring the level of a protein; by monitoring the expression of an autophagic marker; or by monitoring the number of autophagic vacuoles.

### DEFINITIONS

By “mammalian target of rapamycin (mTOR)” is meant a polypeptide sequence having at least 85% or 95% identity to GenBank Accession No. P42345.

By “inhibits mTOR” is meant reduces by at least 10% at least one biological activity associated with mTOR. Exemplary “mTOR biological activities” include mTOR kinase activity, induction of autophagy in cells, the regulation of cell cycle progression, DNA recombination, and DNA damage detection. Compounds that inhibit the phosphorylation of mTor and S6 kinase also inhibit mTOR biological activity.

By “Ras homolog enriched in brain (rheb)” is meant a polypeptide sequence having at least 85% or 95% identity to GenBank Accession No. NP_{—}005605.

By “inhibits rheb” is meant reduces by at least 10% at least one biological activity associated with mTOR. Exemplary “rheb biological activities” include the induction of autophagy, phosphorylation of mTOR, guanine nucleotide binding activity, and GTPase activity.

By “protein conformational disease” is meant a disease or disorder whose pathology is related to the presence of a misfolded protein. In one embodiment, a protein conformational disease is caused when a misfolded protein interferes with the normal biological activity of a cell, tissue, or organ.

By “rapamycin biological activity” is meant enhancement of autophagy, mTOR inhibition, inhibition of T-lymphocyte proliferation, inhibition of lymphokine secretion, inhibition of yeast cell proliferation, enhancement of protein degradation, or any other effect associated with administering rapamycin to a cell or organism.

By “autophagic protein degradation” is meant degradation that occurs substantially by autophagy.

By “analog” is meant a compound that is structurally related to a reference compound and shares essentially the same function as the reference compound. By analog is also meant a derivative or metabolite of a reference compound.

By “enhances” is meant a positive alteration of at least 10%, 15%, 25%, 50%, 75%, or 100%.

By “misfolded protein” is meant a protein having an alteration that affects its tertiary structure relative to a reference protein. Exemplary misfolded proteins include mutant forms of opsin (e.g., P23H opsin), i.e., forms having a sequence alteration relative to an opsin reference sequence (e.g., GenBank Accession Nos. NM_{—}000539 and NP_{—}000530) and mutant forms of human CFTR (e.g., having a ΔF508 mutation), i.e., forms having a sequence alteration relative to a CFTR reference sequence (e.g., GenBank Accession Nos. AAA35680, NP_{—}000483, P13569)

The term “microspheres” refers to substantially spherical colloidal structures having a bioactive substance incorporated therein. The microspheres generally have a size distribution within the range of from about 0.5 μM to about 500 μM. If the constructs are less than one micron in diameter, then the corresponding terms “nanosphere,” may be utilized

By “nanoemulsion” is meant oil-in-water dispersions comprising small lipid structures. In one example, a nanoemulsion comprises an oil phase having droplets with a mean particle size of approximately 0.5 to 5 microns.

By “reduces” is meant a negative alteration of at least 10%, 25%, 50%, 75%, or 100%.

By “selective degradation” is meant degradation that preferentially effects misfolded proteins, such that correctly folded proteins are substantially unaffected. In various embodiments, less than 45%, 35%, 25%, 15%, 10%, or 5% of correctly folded proteins are degraded.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

### BRIEF DESCRIPTION OF THE DRAWINGS

FIGS. 1A-1I show that autophagy causes the selective degradation of misfolded P23H opsin relative to wild-type (WT) opsin. FIGS. 1A, 1C, and 1E are immunoblots showing opsin protein expression in HEK-293 cells stably transfected with wild-type opsin (FIG. 1A) or P23H opsin (FIG. 1C) and treated with rapamycin or amino acid depletion to induce autophagy. In FIG. 1E the P23H opsin expressing cells are further treated with 11-cis retinal. A time-course of induction of autophagy is shown. FIGS. 1B, 1D, and 1E are graphs showing the degradation profile of wild-type opsin (FIG. 1B), P23H opsin (FIG. 1D) and P23H opsin rescued with 11-cis retinal (FIG. 1F) over time. The following conditions are each denoted by the respective symbols: culture media with amino-acids (♦), amino-acid starved (▪), rapamycin (▴) and amino-acid starvation with rapamycin (). FIGS. 1G, 1H, and 1I are immunoblots. FIG. 1G shows dephosphorylation of mTOR during autophagy induction in wild-type and P23H expressing cells. FIGS. 1H and 1I show protein expression of chaperones Bip, calnexin and Hsp70 under autophagic conditions in cells expressing P23H (FIG. 1H) or in cells expressing P23H that were also treated with 11-cis-retinal (FIG. 1I).

FIGS. 2A-2F show that autophagy causes preferential degradation of LF508 over the wild-type. FIGS. 2A and 2C are immunoblots showing HA-tagged CFTR expression in BHK cells stably expressing wild-type CFTR (FIG. 2A) or ΔF508 (FIG. 2C) following amino-acid depletion (lanes 4-6) or rapamycin treatment 50 mM (lanes 7-9) or both (lanes 10-12). FIGS. 2B and 2D are graphs showing the degradation profile of wild-type CFTR FIG. 2B) and ΔF508 (FIG. 2D) over time. The following conditions are each denoted by the respective symbols: culture media with amino-acids (♦), amino-acid starved (▴), rapamycin (▪) and amino-acid starvation with rapamycin (). FIG. 2E is an immunoblot showing dephosphorylation of mTOR following autophagy induction in wild-type CFTR and ΔF508 expressing cells. FIG. 2F shows regulation of chaperones Bip, calnexin, calreticulin and Hsp70 under autophagic conditions in cells expressing wild-type CFTR or ΔF508.

FIGS. 3A-3B are micrographs showing immunofluorescent staining for autophagic markers in HEK-293 cells expressing wild-type opsin (FIG. 3A) or P23H opsin (FIG. 3B). The staining shows that autophagic markers colocalize with misfolded aggregates of P23H opsin. Colocalization of opsin (middle panel) with autophagy markers (left panel) Atg7, LC3 and LAMP-1 is shown under normal or amino acid depleted conditions.

FIGS. 4A-B are micrographs showing immunofluorescent staining for autophagy markers and CFTR in BHK cells expressing wild-type CFTR (FIG. 4A) or ΔF508 (FIG. 4B). The autophagic markers colocalize with ΔF508 CFTR protein retained in the ER. Colocalization of HA-tagged CFTR (middle panel) with autophagy markers (left panel) Atg7, LC3 and LAMP-1 is shown under normal or amino acid depleted conditions.

FIGS. 5A-5C shows three electron micrographs of cells. FIG. 5A shows P23H aggregates in cells stained for rhodopsin and immunogold labeled. FIGS. 5B and C show lysosomes and autophagic vacuoles in autophagic cells.

FIG. 6 is a bar graph showing that rapamycin treatment enhances retinal function in a P23H mouse model of retinitis pigmentosa. Control-P23H1 and P23H2 are transgenic mice expressing one copy of P23H opsin. Rap-P23HA and B are transgenic mice expressing one copy of P23H opsin and treated with rapamycin. WT-1, 2, and 3 are wild-type control mice. Rap-WTA and B are wild-type control mice that received rapamycin. Each bar represents an ERG assay of a single mouse.

FIG. 7 is a bar graph showing that rapamycin treatment enhances retinal function in a mouse model of macular degeneration. Each bar represents an ERG assay of a single mouse.

FIG. 8 is a Western blot showing P231-1 protein degradation in P23H expressing cells treated with the farnesyl protein transferase inhibitor FTI277. A Western blot for a tubulin is shown below as a loading control. The term “Fed” denotes culture condition with amino-acid and serum containing media; the term “F+R” denotes fed and treated with rapamycin; the term “F+FTI277” denotes Fed and treated with 10 μM or 50 μM of FTI277. A Western blot probed with tubulin is shown below as a control for protein loading.

FIG. 9A-9B show that FTI-277 treatment results in P23H opsin degradation. FIG. 9A is an immunoblot showing P23H opsin levels following Rheb inhibition with FTI-277 over the time course of twelve hours. P23H opsin-expressing cells were treated with either 10 μM (lanes 8-10) or 50 μM (lanes 11-13) FTI-277. Rapamycin (lanes 5-7) treatment was used as a positive control. Amino acid and serum fed controls were also used (lanes 2-4). Tubulin serves as a loading control. FIG. 9B is a graph showing degradation profiles based on the pixel intensities of the bands on the immunoblot comparing rapamycin and FTI-277 (50 μM) treatment in P23H opsin expressing cells at two hours (h), six hours and twelve hours.

FIG. 10 is a set of immunoblots showing chaperones calnexin, calreticulin, Hsp70 and Hsp90 levels. This work analyses the effect of FTI-277 treatment on both unfolded protein response and heat shock response in a time-dependent manner, and shows that autophagy is exclusive of UPR and HSR.

FIGS. 11A-11C are a set of immunoblots showing that FTI-277 blocks phosphorylation of S6K and mTOR. FIG. 11A shows that the phosphorylation state of S6K was determined following treatment with rapamycin and FTI-277 within two hours. FIG. 11B shows that the phosphorylation state of mTOR was determined using rapamycin, FTI-277 and FTI-277 in combination with amino acid and serum starvation over a time course of twelve hours.

FIGS. 12A-12C are a series of micrographs showing the immunocolocalization of autophagosome markers following FTI-277 treatment. FIG. 12A shows Atg7 staining and FIG. 12B shows Atg8 staining. These markers were used to observe colocalization with P23H opsin aggregates following treatment with FTI-277. Amino acid fed and rapamycin-treated cells were used as controls. FIG. 12C shows confocal imaging of cells treated with FTI-277. Confocal imaging clearly indicates the intracellular localization between autophagosome markers Atg7 and Atg8 and P23H opsin aggregates. Autophagosome antibodies are TRITC (left) labeled and opsin is FITC (right) labeled.

FIGS. 13A-13C show the cell's autophagic response to FTI-277 treatment. FIG. 13A shows that lysotracker was used as a marker to observe upregulation of the lysosomal pathway in cells following FTI-277 treatment. FIG. 13B shows an electron micrograph ultrastructure analysis, which was performed following treatment of cells with FTI-277 for six hours. The cells were processed for cMPase cytochemistry to visualize lysosomes as well as autolysosomes. FIG. 13C is a graph which shows the results of a morphometric analysis performed at two hours and six hours following FTI-277 treatment. This compares autophagic induction upon treatment of FTI-277 with that of amino acid and serum fed controls.

FIG. 14 is a confocal analysis showing overexpression of GFP-LC3 in HuH7 cells following FTI-277 treatment.

FIGS. 15A and 15B show FTI-277 induced autophagy. FIG. 15A shows two immunoblots performed following treatment of the cells with the autophagy inhibitor 3MA. 3MA inhibited P23H opsin degradation induced by FTI-277. Amino-acid and serum starved cells (Top lane) were used as a control to observe accumulation of P23H opsin at twenty-four hours. Similarly, FTI-277 treatment in the presence of 3MA (light gray bars) prevented degradation of P2311 opsin. Maximum accumulation of opsin was observed at twelve hours in 3MA treated cells compared to FTI-277 treatment alone (dark gray bars). FIG. 15B shows P23H opsin expressing cells treated with proteasome inhibitor, MG132, under fed (light gray bars) and FTI-277 treated (dark gray bars) conditions for twelve hours showed that MG132 did not interfere with the degradation of misfolded P23H opsin during starvation.

FIG. 16 is a schematic diagram of the insulin/TOR/S6K pathway showing the sites of inhibition by FTI-277 and rapamycin. Activation of PI3kinases leads to phosphorylation of Akt. TSC1/2 act as a GAP to activate Rheb, which in turn phosphorylates mTOR. Inhibition of mTOR leads to induction of autophagy in cells. Potential inhibition of Rheb by FTI-277 induces autophagy similar to inhibition of mTOR by rapamycin.

### DETAILED DESCRIPTION OF THE INVENTION

The invention features compositions and methods that are useful for enhancing the degradation of misfolded proteins in vivo. In general, the invention is based on the discovery that compounds of the invention (e.g., rapamycin, FTI-277, analogs and variants thereof) enhance the degradation of mutant proteins. Advantageously, mutant proteins are specifically degraded, while levels of the respective wild-type forms remain largely unchanged. Misfolded proteins can interfere with normal cell function, and can cause cytotoxicity, resulting in a human Protein Conformational Disease (PCD). PCDs include α1-antitrypsin deficiency, cystic fibrosis, Huntington's disease, Parkinson's disease, Alzheimer's disease, nephrogenic diabetes insipidus, cancer, and prion-related disorders (e.g., Jacob-Creutzfeld disease). The compositions and methods of the invention are particularly useful for the prevention or treatment of ocular PCDs, including retinitis pigmentosa, age-related macular degeneration (wet and dry forms), glaucoma, corneal dystrophies, retinoschises, Stargardt's disease, autosomal dominant druzen, Best's macular dystrophy, and corneal dystrophies. Compositions of the invention can be used to treat the PCD, to slow the death of affected cells, to relieve symptoms caused by the PCD, or to prevent a PCD from being initiated in the first place.

### Autophagy Enhancers

Autophagy is an evolutionarily conserved mechanism for the degradation of cellular components in the cytoplasm, and serves as a cell survival mechanism in starving cells. During autophagy pieces of cytoplasm become encapsulated by cellular membranes, forming autophagic vacuoles that eventually fuse with lysosomes to have their contents degraded. Autophagy enhancers may be used independently or in combination with 11-cis-retinal, 9-cis-retinal, or a 7-ring locked isomer of 11-cis-retinal for the treatment of a PCD. The autophagy enhancer rapamycin is particularly useful for the treatment of retinitis pigmentosa and other ocular diseases as well as for the treatment of cystic fibrosis and other disorders related to misfolded proteins or protein aggregation. Autophagy enhancers useful in the methods of the invention include, but are not limited to, rapamycin, FTI-277, and salts or analogs thereof.

### Rapamycin

Rapamycin (Rapamune®, sirolimus, Wyeth) is a cyclic lactone produced by Steptomyces hygroscopicus. Rapamycin is an immunosuppressive drug that inhibits T-lymphocyte activation and proliferation. Rapamycin binds to and inhibits the mammalian target of rapamycin (mTOR), a kinase that is required for cell cycle progression. Inhibition of mTOR kinase activity blocks T-lymphocyte proliferation and lymphokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Other rapamycin analogs include CCl-779, (Wyeth Ayerst), tacrolimus, pimecrolimus, AP20840 (Ariad Pharmaceutics), AP23841 (Ariad Pharmaceutics), and ABT-578 (Abbott Laboratories), SAR943 (32-deoxorapamycin, Eynott et al., Immunology. 109(3):461-7, 2003), and everolimus (SDZ RAD). Everolimus (40-O-(2-hydroxyethyl)rapamycin (CERTICAN, Novartis) is an immunosuppressive macrolide that is structurally related to rapamycin. Additional rapamycin analogs are described in U.S. Pat. Nos. 5,202,332 and 5,169,851.

Rapamycin is currently available for oral administration in liquid and tablet formulations. RAPAMITNE™ liquid contains 1 mg/mL rapamycin that is diluted in water or orange juice prior to administration. Tablets containing 1 or 2 mg of rapamycin are also available. Rapamycin is preferably given once daily. It is absorbed rapidly and completely after oral administration. Typically, patient dosage of rapamycin varies according to the patient's condition, but some standard recommended dosages are provided below. The initial loading dose for rapamycin is 6 mg. Subsequent maintenance doses of 2 mg/day are typical. Alternatively, a loading dose of 3 mg, 5 mg, 10 mg, 15 mg, 20 mg, or 25 mg can be used with a 1 mg, 3 mg, 5 mg, 7 mg, or 10 mg per day maintenance dose. In patients weighing less than 40 kg, rapamycin dosages are typically adjusted based on body surface area; generally a 3 mg/m²/day loading dose and a 1 mg/m²/day maintenance dose is used.

### Farnesyl Transferase Inhibitors

Farnesyl transferase inhibitors inhibit farnesylation, which is a post-translational modification of proteins that increases the hydrophobicity of the modified protein causing it to localize at the surface of the cell membrane. This localization to the cell membrane is typically necessary for the normal function of farnesylated proteins. Farnesyl acceptor moieties have been characterized in various proteins as a four amino acid sequence found at the carboxy terminus of target proteins. This four amino acid sequence has been characterized as —C-A-A-X, wherein “C” is a cysteine residue, “A” refers to any aliphatic amino acid, and “X” refers to any amino acid. Farnesyltransferase inhibitors (FTIs), such as FTI-277, inhibit the post-translational lipid modification of Ras and other farnesylated proteins, such as Rheb. As reported in more detail below, FTI-277 is an exemplary farnesyl transferase inhibitor that induces autophagy in cells by inactivating mTOR. mTOR negatively controls autophagy as a downstream target for AKT/PKB in response to amino acids. Based in part on this discovery, other agents that inhibit farnesyl transferases are also useful in the methods of the invention.

Farnesyl transferase inhibitors useful in the methods of the invention are known in the art and are described, for example, in U.S. Pat. Nos. 7,022,704, 6,936,431, 6,800,636, 6,790,633, 6,740,661, 6,737,410, 6,576,639, 6,528,523, 6,498,152, 6,440,974, 6,432,959, 6,410,541, 6,399,615, 6,372,747, 6,362,188; in other embodiments, 3-mercaptopyrrolidines FTIs are described, for example, in U.S. Pat. No. 6,946,468; 5-substituted tetralones FTIs are described, for example, in U.S. Pat. No. 6,943,183; bicyclic inhibitors are described in U.S. Pat. No. 6,528,535; and triazoles as farnesyl transferase inhibitors are described, for example, in 20050234117. Other exemplary FTIs useful in the methods of the invention are described in U.S. Patent Application Publication Nos: 20060079530, 20050148609, 20050059672, and 20050020516; and in the following scientific publications: Santucci et al., Cancer Control 10:384-387, 2003; Mégnin-Chanet et al., BMC Pharmacology, 2:2, 2002, BMS-214662; and Appels et al., (The Oncologist, 10: 565-578, 2005). Exemplary farnesyl transferase inhibitors include, but are not limited to, R115777, GGTI-2166, BMS-214664, which are described by Santucci et al., Cancer Control 10:384-387, 2003; RPR-130401, which is described by Mégnin-Chanet et al., BMC Pharmacology, 2:2, 2002; BMS-214662 (Bristol-Myers Squibb, Princeton, N.J.); L778123 (Merck & Co., Inc., Whitehouse Station, N.J.); tipifarnib (experimental name, R115777; Zarestra™; Ortho Biotech Products, L.P., Bridgewater, N.J.); lonafarnib (experimental name, SCH66336; Sarasar™; Schering-Plough Corporation, Kenilworth, N.J.); FTI-277 (Calbiochem, EMD Biosciences, San Diego); and L744832 (Biomol International L.P., Plymouth Meeting, Pa.).

Suggested clinical doses of FTIs are typically between 100 ug and 10,000 mg daily. Administration may be by any method known in the art. In particular, tipifarnib is administered in doses of 150, 200, 300, 400, 500, and 600 mg orally twice daily for 21 days. Lonafarnib is administered in 100, 200, 300, and 400 mg doses orally twice daily on a continuous regimen. BMS-214662 is typically administered intravenously in a 1 hour infusion once every 3 weeks at 100 mg/m², 200 mg/m², at 275 mg/m², and 300 mg/m² for a 24-hour infusion; alternatively, BMS-214662 is administered at 300 mg/m² on a weekly schedule and 102 mg/m² on a weekly schedule. Administration of BMS-214662 on a daily basis at 81 mg/m² is also useful in the methods of the invention. Other modes of administering FTIs are known in the art, and are describe, for example, by Appels et al., (The Oncologist, 10: 565-578, 2005).

### Ocular Protein Conformational Disorders

Compositions of the invention are particularly useful for the treatment of virtually any ocular protein conformational disorder (PCD). Such disorders are characterized by the accumulation of misfolded proteins as protein aggregates or fibrils within the eye. The compositions of the invention selectively enhance the degradation of misfolded proteins while leaving correctly folded protein levels largely unaffected. Retinitis pigmentosa is an exemplary ocular PCD that is associated with the misfolding of an opsin (e.g., P23H opsin) (GenBank Accession Nos. NM_{—}000539 and NP_{—}000530), as well as with mutations in carbonic anhydrase IV (CA4)) (GenBank Accession Nos. NM_{—}000717 and NP_{—}000708) (Rebello et al., Proc Natl Acad Sci USA. 2004 Apr. 27; 101(17):6617-22). CA4 is a glycosylphosphatidylinositol-anchored protein that is highly expressed in the choriocapillaris of the human eye. An R14W mutation causes the CA4 protein to be incorrectly folded and patients carrying this mutation suffer from autosomal dominant retinitis pigmentosa. Compositions of the invention that enhance the degradation of a mutant form of the CA4 polypeptide are useful for the treatment of autosomal dominant retinitis pigmentosa associated with mutations in the CA4 polypeptide.

X-linked juvenile retinoschisis (RS) is another ocular PCD. RS is a common cause of juvenile macular degeneration in males. Mutations in RS1 (NM_{—}000330, NM_{—}000321), or retinoschesin, are responsible for X-linked retinoschisis, a common, early-onset macular degeneration in males that results in a splitting of the inner layers of the retina and severe loss in vision. Mutations in RS1 disrupt protein folding (J Biol Chem. 2005 Mar. 18; 280(11):10721-30). Compositions of the invention that enhance the degradation of a mutant form of RS1 are useful for the treatment of retinoschisis.

Glaucoma is an ocular PCD that is associated with mutations in myocilin. Myocilin is a secreted glycoprotein of unknown function that is ubiquitously expressed in many human organs, including the eye. Mutations in this the myocilin protein cause one form of glaucoma, a leading cause of blindness worldwide. Mutant myocilins accumulate in the endoplasmic reticulum of transfected cells as insoluble aggregates (Aroca-Aguilar et al., Biol. Chem. 2005 Jun. 3; 280(22):21043-51; GenBank Accession Nos. NM_{—}000261 and NP_{—}000252). Compositions of the invention that enhance the degradation of a mutant form of myocilin are useful for the treatment of myocilin-associated glaucoma.

Stargardt-like macular degeneration is an ocular PCD that is associated with mutations in ELOVL4. ELOVL4 (Elongation of very long chain fatty acids 4) is a member of the ELO family of proteins involved in the biosynthesis of very long chain fatty acids. Mutations in ELOVL4 have been identified in patients with autosomal dominant Stargardt-like macular degeneration (STGD3/adMD). ELOVL4 mutant proteins accumulate as large aggregates in transfected cells (Grayson et al., J Biol Chem. 2005 Jul. 21; Epub) (GenBank Accession Nos. NM_{—}022726 and NP_{—}073563). Compositions of the invention that enhance the degradation of a mutant form ELOVL4 are useful for the treatment of Stargardt-like macular degeneration.

Malattia Leventinese (ML) and Doyne honeycomb retinal dystrophy (DHRD) refer to two autosomal dominant PCDs that are characterized by yellow-white deposits known as drusen that accumulate beneath the retinal pigment epithelium (RPE). EFEMP1 has a role in retinal drusen formation and is involved in the etiology of macular degeneration (Stone et al., Nat. Genet. 1999 June; 22(2):199-202) (GenBank Accession Nos NM_{—}004105 and NP_{—}004096). Mutant EFEMP1 is misfolded and retained within cells. Compositions of the invention that enhance the degradation of a mutant form of EFEMP1 are useful for the treatment of autosomal dominant drusen.

Best's macular dystrophy is an autosomal dominant PCD that is caused by mutations in VMD2 (hBEST1), which encodes Bestrophin, a Cl(−) channels (Gomez et al., DNA Seq. 2001 December; 12(5-6):431-5) (GenBank Accession Nos: NM_{—}004183 and NP_{—}004174). Mutations in bestrophin likely cause protein misfolding. Compositions of the invention that enhance the degradation of a mutant form of correctly folded bestrophin are useful for the treatment of Best's macular dystrophy.

5q31-linked corneal dystrophies are autosomal dominant PCDs that are characterized by age-dependent progressive accumulation of protein deposits in the cornea followed by visual impairment. Mutations in the BIGH3 gene (GenBank Accession No: NM_{—}000358), also termed TGFBI (transforming growth factor-β-induced) were found to be responsible for this entire group of conditions. Substitutions at the Arg-124 as well as other residues result in cornea-specific deposition of the encoded protein (GenBank Accession No. NP_{—}000349) via distinct aggregation pathways that involve altered turnover of the protein in corneal tissue. Compositions of the invention that enhance the degradation of a mutant form of correctly folded TGFBI protein are useful for the treatment of 5q31-linked corneal dystrophies.

### Therapeutic Methods

The present invention provides methods of treating a PCD disease and/or disorders or symptoms thereof (e.g., cytotoxicity) by selectively enhancing the degradation of a misfolded protein. The methods comprise administering a therapeutically effective amount of a pharmaceutical composition comprising a compound (e.g., an mTOR inhibitor, such as rapamycin, a farnesyl transferase inhibitor, such as FTI-277) described herein to a subject (e.g., a mammal such as a human). Thus, one embodiment is a method of treating a subject suffering from or susceptible to a protein conformation disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention, which include prophylactic treatment, in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which protein folding (including misfolding) may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with protein folding (including misfolding), in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Pharmaceutical Compositions

The present invention features pharmaceutical preparations comprising compounds together with pharmaceutically acceptable carriers, where the compounds provide for the selective degradation of a misfolded protein. Such preparations have both therapeutic and prophylactic applications. In one embodiment, a pharmaceutical composition includes 11-cis-retinal or 9-cis-retinal in combination with the compound that enhances degradation of a misfolded protein. The 11-cis-retinal or 9-cis-retinal and the compound are formulated together or separately. Compounds of the invention may be administered as part of a pharmaceutical composition. The compositions should be sterile and contain a therapeutically effective amount of the polypeptides in a unit of weight or volume suitable for administration to a subject. The compositions and combinations of the invention can be part of a pharmaceutical pack, where each of the compounds is present in individual dosage amounts.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein. See, e.g., Alexander, J. et al. Journal of Medicinal Chemistry 1988, 31, 318-322; Bundgaard, H. Design of Prodrugs; Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development; Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development; 2 ed.; Overseas Publ.: Amsterdam, 1996; pp 351-385; Pitman, I. H. Medicinal Research Reviews 1981, 1, 189-214; Sinkula, A. A.; Yalkowsky. Journal of Pharmaceutical Sciences 1975, 64, 181-210; Verbiscar, A. J.; Abood, L. G Journal of Medicinal Chemistry 1970, 13, 1176-1179; Stella, V. J.; Himmelstein, K. J. Journal of Medicinal Chemistry 1980, 23, 1275-1282; Bodor, N.; Kaminski, J. J. Annual Reports in Medicinal Chemistry 1987, 22, 303-313.

The compounds of this invention may be modified by appending appropriate functionalities to enhance selective biological properties. Such modifications are known in the art and include those which increase biological penetration into a given biological compartment (e.g., blood, lymphatic system, nervous system), increase oral availability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

Pharmaceutical compositions of the invention to be used for prophylactic or therapeutic administration should be sterile. Sterility is readily accomplished by filtration through sterile filtration membranes (e.g., 0.2 μm membranes), by gamma irradiation, or any other suitable means known to those skilled in the art. Therapeutic polypeptide compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution.

The compounds may be combined, optionally, with a pharmaceutically acceptable excipient. The term “pharmaceutically-acceptable excipient” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with the molecules of the present invention, and with each other, in a manner such that there is no interaction that would substantially impair the desired pharmaceutical efficacy.

The excipient preferably contains minor amounts of additives such as substances that enhance isotonicity and chemical stability. Such materials are non-toxic to recipients at the dosages and concentrations employed, and include buffers such as phosphate, citrate, succinate, acetate, lactate, tartrate, and other organic acids or their salts; tris-hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be included, such as stabilizers, anti-microbials, inert gases, fluid and nutrient replenishers (i.e., Ringer's dextrose), electrolyte replenishers, and the like, which can be present in conventional amounts.

The compositions, as described above, can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject having a protein conformation disease or disorder, an effective amount is sufficient to increase the level of a correctly folded protein in a cell. With respect to a subject having a disease or disorder related to a misfolded protein, an effective amount is an amount sufficient to stabilize, slow, or reduce the a symptom associated with a pathology. Generally, doses of the compounds of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of a composition of the present invention.

A variety of administration routes are available. The methods of the invention, generally speaking, may be practiced using any mode of administration that is medically acceptable, meaning any mode that produces effective levels of the active compounds without causing clinically unacceptable adverse effects. In one preferred embodiment, a composition of the invention is administered intraocularly. Other modes of administration include oral, rectal, topical, intraocular, buccal, intravaginal, intracistemal, intracerebroventricular, intratracheal, nasal, transdermal, within/on implants, or parenteral routes. The term “parenteral” includes subcutaneous, intrathecal, intravenous, intramuscular, intraperitoneal, or infusion. Compositions comprising a composition of the invention can be added to a physiological fluid, such as to the intravitreal humor. For CNS administration, a variety of techniques are available for promoting transfer of the therapeutic across the blood brain barrier including disruption by surgery or injection, drugs which transiently open adhesion contact between the CNS vasculature endothelial cells, and compounds that facilitate translocation through such cells. Oral administration can be preferred for prophylactic treatment because of the convenience to the patient as well as the dosing schedule.

Pharmaceutical compositions of the invention can optionally further contain one or more additional proteins as desired, including plasma proteins, proteases, and other biological material, so long as it does not cause adverse effects upon administration to a subject. Suitable proteins or biological material may be obtained from human or mammalian plasma by any of the purification methods known and available to those skilled in the art; from supernatants, extracts, or lysates of recombinant tissue culture, viruses, yeast, bacteria, or the like that contain a gene that expresses a human or mammalian plasma protein which has been introduced according to standard recombinant DNA techniques; or from the fluids (e.g., blood, milk, lymph, urine or the like) or transgenic animals that contain a gene that expresses a human plasma protein which has been introduced according to standard transgenic techniques.

Pharmaceutical compositions of the invention can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g, tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

Compositions comprising a compound of the present invention can contain multivalent metal ions, such as calcium ions, magnesium ions and/or manganese ions. Any multivalent metal ion that helps stabilizes the composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (s) contained therein. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PPG 2000, PPG 3000 and PPG 4000.

Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the compound contained therein. Preferably, the non-aqueous liquid in such a formulation is a hydrophilic liquid. Illustrative examples of suitable non-aqueous liquids include: glycerol; DMSO; PMS; ethylene glycols, such as PEG 200, PEG 300, and PEG 400; and propylene glycols, such as PPG 425, PPG 725, PPG 1000, PPG 2000, PPG 3000 and PPG 4000.

Suitable stable formulations can permit storage of the active agents in a frozen or an unfrozen liquid state. Stable liquid formulations can be stored at a temperature of at least −70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

Other delivery systems can include time-release, delayed release or sustained release delivery systems. Such systems can avoid repeated administrations of compositions of the invention, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(−)-3-hydroxybutyric acid (European Patent No. 133, 988), copolymers of L-glutamic acid and gamma-ethyl-L-glutamate (Sidman, K. R. et al., Biopolymers 22: 547-556), poly (2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono- di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like. Specific examples include, but are not limited to: (a) erosional systems in which the agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88, 046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/0307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (where an agent is dispersed throughout a solid polymeric matrix) or a microcapsule (where an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and composition are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. E., et al., Biotechnol. Bioeng., 52: 96-101; Mathiowitz, E., et al., Nature 386: 410-414.

Both non-biodegradable and biodegradable polymeric matrices can be used to deliver the compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

### Methods of Delivery to the Pulmonary Epithelia

In certain embodiments, such as for the treatment of cystic fibrosis, where it is desirable to administer a compound of the invention directly to the pulmonary epithelia, a desirable route of administration can be by pulmonary aerosol. Drugs intended for pulmonary delivery can be administered as aqueous formulations, as suspensions or solutions in halogenated hydrocarbon propellants, or as dry powders. Aqueous formulations must be aerosolized by liquid nebulizers employing either hydraulic or ultrasonic atomization, propellant-based systems require suitable pressurized metered-dose inhalers, and dry powders require dry powder inhaler devices which are capable of dispersing the drug substance effectively. For aqueous and other non-pressurized liquid systems, a variety of nebulizers (including small volume nebulizers) are available to aerosolize the formulations. Compressor-driven nebulizers incorporate jet technology and use compressed air to generate the liquid aerosol. Ultrasonic nebulizers rely on mechanical energy in the form of vibration of a piezoelectric crystal to generate respirable liquid droplets. A propellant driven inhaler releases a metered dose of medicine upon each actuation. The medicine is formulated as a suspension or solution of a drug substance in a suitable propellant. Dry powder inhalers normally rely upon a burst of inspired air that is drawn through the unit to deliver a drug dosage. Such devices are described in, for example, U.S. Pat. Nos. 4,807,814 and 5,785,049.

Pulmonary drug delivery is accomplished by inhalation of an aerosol through the mouth and throat. Particles having diameters of about 2 to about 5 microns are small enough to reach the upper- to mid-pulmonary region (conducting airways), but are too large to reach the alveoli. Even smaller particles, i.e., about 0.5 to about 2 microns, are capable of reaching the alveolar region. Particles having diameters smaller than about 0.5 microns can also be deposited in the alveolar region by sedimentation, although very small particles may be exhaled. Techniques for preparing aerosol delivery systems are well known to those of skill in the art. See U.S. Pat. Nos. 4,512,341, 4,566,452, 4,746,067, 5,008,048, 6,796,303, and U.S. Patent Publication No. 20020102294. Those of skill in the art can readily modify the various parameters and conditions for producing pulmonary aerosols without resorting to undue experimentation.

### Methods of Ocular Delivery

The compositions of the invention (e.g., an autophagy enhancer, an mTOR inhibitor, such as rapamycin, a farnesyl transferase inhibitor, such as FTI-277) are particularly suitable for treating ocular protein conformation diseases, such as glaucoma, retinitis pigmentosa, age-related macular degeneration, glaucoma, corneal dystrophies, retinoschises, Stargardt's disease, autosomal dominant druzen, and Best's macular dystrophy.

In one approach, the compositions of the invention are administered through an ocular device suitable for direct implantation into the vitreous of the eye. The compositions of the invention may be provided in sustained release compositions, such as those described in, for example, U.S. Pat. Nos. 5,672,659 and 5,595,760. Such devices are found to provide sustained controlled release of various compositions to treat the eye without risk of detrimental local and systemic side effects. An object of the present ocular method of delivery is to maximize the amount of drug contained in an intraocular device or implant while minimizing its size in order to prolong the duration of the implant. See, e.g., U.S. Pat. Nos. 5,378,475; 6,375,972, and 6,756,058 and U.S. Publications 20050096290 and 200501269448. Such implants may be biodegradable and/or biocompatible implants, or may be non-biodegradable implants. Biodegradable ocular implants are described, for example, in U.S. Patent Publication No. 20050048099. The implants may be permeable or impermeable to the active agent, and may be inserted into a chamber of the eye, such as the anterior or posterior chambers or may be implanted in the schlera, transchoroidal space, or an avascularized region exterior to the vitreous. Alternatively, a contact lens that acts as a depot for compositions of the invention may also be used for drug delivery.

In a preferred embodiment, the implant may be positioned over an avascular region, such as on the sclera, so as to allow for transcleral diffusion of the drug to the desired site of treatment, e.g. the intraocular space and macula of the eye. Furthermore, the site of transcleral diffusion is preferably in proximity to the macula. Examples of implants for delivery of an a composition include, but are not limited to, the devices described in U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146,366; 6,251,090; and 6,299,895, and in WO 01/30323 and WO 01/28474, all of which are incorporated herein by reference.

Examples include, but are not limited to the following: a sustained release drug delivery system comprising an inner reservoir comprising an effective amount of an agent effective in obtaining a desired local or systemic physiological or pharmacological effect, an inner tube impermeable to the passage of the agent, the inner tube having first and second ends and covering at least a portion of the inner reservoir, the inner tube sized and formed of a material so that the inner tube is capable of supporting its own weight, an impermeable member positioned at the inner tube first end, the impermeable member preventing passage of the agent out of the reservoir through the inner tube first end, and a permeable member positioned at the inner tube second end, the permeable member allowing diffusion of the agent out of the reservoir through the inner tube second end; a method for administering a compound of the invention to a segment of an eye, the method comprising the step of implanting a sustained release device to deliver the compound of the invention to the vitreous of the eye or an implantable, sustained release device for administering a compound of the invention to a segment of an eye; a sustained release drug delivery device comprising: a) a drug core comprising a therapeutically effective amount of at least one first agent effective in obtaining a diagnostic effect or effective in obtaining a desired local or systemic physiological or pharmacological effect; b) at least one unitary cup essentially impermeable to the passage of the agent that surrounds and defines an internal compartment to accept the drug core, the unitary cup comprising an open top end with at least one recessed groove around at least some portion of the open top end of the unitary cup; c) a permeable plug which is permeable to the passage of the agent, the permeable plug is positioned at the open top end of the unitary cup where the groove interacts with the permeable plug holding it in position and closing the open top end, the permeable plug allowing passage of the agent out of the drug core, through the permeable plug, and out the open top end of the unitary cup; and d) at least one second agent effective in obtaining a diagnostic effect or effective in obtaining a desired local or systemic physiological or pharmacological effect; or a sustained release drug delivery device comprising: an inner core comprising an effective amount of an agent having a desired solubility and a polymer coating layer, the polymer layer being permeable to the agent, where the polymer coating layer completely covers the inner core.

Other approaches for ocular delivery include the use of liposomes to target a compound of the present invention to the eye, and preferably to retinal pigment epithelial cells and/or Bruch's membrane. For example, the compound may be complexed with liposomes in the manner described above, and this compound/liposome complex injected into patients with an ocular PCD, using intravenous injection to direct the compound to the desired ocular tissue or cell. Directly injecting the liposome complex into the proximity of the retinal pigment epithelial cells or Bruch's membrane can also provide for targeting of the complex with some forms of ocular PCD. In a specific embodiment, the compound is administered via intra-ocular sustained delivery (such as VITRASERT or ENVISION). In a specific embodiment, the compound is delivered by posterior subtenons injection. In another specific embodiment, microemulsion particles containing the compositions of the invention are delivered to ocular tissue to take up lipid from Bruch's membrane, retinal pigment epithelial cells, or both.

Nanoparticles are a colloidal carrier system that has been shown to improve the efficacy of the encapsulated drug by prolonging the serum half-life. Polyalkylcyanoacrylates (PACAs) nanoparticles are a polymer colloidal drug delivery system that is in clinical development, as described by Stella et al., J. Pharm. Sci., 2000. 89: p. 1452-1464; Brigger et al., Int. J. Pharm., 2001. 214: p. 37-42; Calvo et al., Pharm. Res., 2001. 18: p. 1157-1166; and Li et al., Biol. Pharm. Bull., 2001. 24: p. 662-665. Biodegradable poly (hydroxyl acids), such as the copolymers of poly (lactic acid) (PLA) and poly (lactic-co-glycolide) (PLGA) are being extensively used in biomedical applications and have received FDA approval for certain clinical applications. In addition, PEG-PLGA nanoparticles have many desirable carrier features including (i) that the agent to be encapsulated comprises a reasonably high weight fraction (loading) of the total carrier system; (ii) that the amount of agent used in the first step of the encapsulation process is incorporated into the final carrier (entrapment efficiency) at a reasonably high level; (iii) that the carrier have the ability to be freeze-dried and reconstituted in solution without aggregation; (iv) that the carrier be biodegradable; (v) that the carrier system be of small size; and (vi) that the carrier enhance the particles persistence.

Nanoparticles are synthesized using virtually any biodegradable shell known in the art. In one embodiment, a polymer, such as poly (lactic-acid) (PLA) or poly (lactic-co-glycolic acid) (PLGA) is used. Such polymers are biocompatible and biodegradable, and are subject to modifications that desirably increase the photochemical efficacy and circulation lifetime of the nanoparticle. In one embodiment, the polymer is modified with a terminal carboxylic acid group (COOH) that increases the negative charge of the particle and thus limits the interaction with negatively charge nucleic acid aptamers. Nanoparticles are also modified with polyethylene glycol (PEG), which also increases the half-life and stability of the particles in circulation. Alternatively, the COOH group is converted to an N-hydroxysuccinimide (NHS) ester for covalent conjugation to amine-modified aptamers.

Biocompatible polymers useful in the composition and methods of the invention include, but are not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, allyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetage phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), poly(octadecl acrylate) and combinations of any of these. In one embodiment, the nanoparticles of the invention include PEG-PLGA polymers.

Compositions of the invention may also be delivered topically. For topical delivery, the compositions are provided in any pharmaceutically acceptable excipient that is approved for ocular delivery. Preferably, the composition is delivered in drop form to the surface of the eye. For some application, the delivery of the composition relies on the diffusion of the compounds through the cornea to the interior of the eye.

Those of skill in the art will recognize that the best treatment regimens for using compounds of the present invention to treat an ocular PCD can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained from the initial clinical trials and the needs of a particular patient.

Human dosage amounts for compositions of the invention (e.g., an autophagy enhancer, an mTOR inhibitor, such as rapamycin, a farnesyl transferase inhibitor, such as FTI-277) can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Where rapamycin is used dosages of 1 mg, 2 mg, 3 mg, 5 mg, 7 mg, 10 mg, 15 mg, 20 mg, or 25 mg can be used per day. Of course, this dosage amount may be adjusted upward or downward, as is routinely done in such treatment protocols, depending on the results of the initial clinical trials and the needs of a particular patient.

### Screening Assays

As discussed herein, misfolded proteins often interfere with the normal biological function of cells and cause PCD. In many cases, the accumulation of misfolded proteins in protein aggregates causes cellular damage and cytotoxicity. Useful compounds enhance the degradation of such proteins, thus ameliorating cytotoxicity. Any number of methods are available for carrying out screening assays to identify such compounds. In one approach, a mutant protein that fails to adopt a wild-type protein conformation is expressed in a cell (e.g., a cell in vitro or in vivo); the cell is contacted with a candidate compound; and the effect of the compound on autophagy is assayed using any method known in the art or described herein. A compound that enhances autophagy is identified by measuring a decrease in the level of a misfolded protein, in measuring a decrease in cytotoxicity, by measuring an increase in the presence of autophagic vacuoles, or by measuring an increase in the level of an autophagic marker (e.g., dephosphorylated mTOR or S6 kinase) using any standard method (e.g., immunoassay). A compound that reduces the amount of misfolded protein present in the contacted cell relative to a control cell that was not contacted with the compound, is considered useful in the methods of the invention. A decrease in the amount of a misfolded protein is assayed, for example, by measuring a decrease in intracellular protein aggregation, by measuring a decrease in cytotoxicity, or by measuring a decrease in the level of the protein. Preferably, the misfolded protein is selectively degraded. In a related approach, the screen is carried out in the presence of rapamycin, FTI-277, 11-cis-retinal, 9-cis-retinal, or an analog or derivative thereof. Useful compounds decrease the amount of misfolded protein by at least 10%, 15%, or 20%, or preferably by 25%, 50%, or 75%; or most preferably by at least 100%, 200%, 300% or even 400%.

If desired, the efficacy of the identified compound is assayed in an animal model having a PCD (e.g., an animal model of retinitis pigmentosa, cystic fibrosis, Huntington's disease, Parkinson's disease, Alzheimer's disease, nephrogenic diabetes insipidus, cancer (e.g., cancer related to p53 mutations), and prion-related disorders (e.g., Jacob-Creutzfeld disease)).

### Screening for Enhancers of Rapamycin Activity

The invention is directed to methods of enhancing autophagy for the treatment of a PCD. Compounds that enhance rapamycin or FTI-277 biological activity are expected to enhance the degradation of misfolded proteins. Accordingly, compounds identified as enhancing a biological effect of rapamycin or FTI-277 are useful in the methods of the invention. In one embodiment, small-molecules that enhance a rapamycin biological activity are identified using a small-molecule target identification strategy in yeast cells. Rapamycin inhibits the growth of wild-type yeast cells. Compounds that enhance the inhibitory effect on yeast cell growth can be identified using routine methods, such as a chemical genetic modifier screen. Such screens are known in the art and are described, for example, by Huang et al., PNAS 101: 16594-16599, 2004. Rapamycin treatment induces a state reminiscent of the nutrient starvation response, often resulting in growth inhibition. Using a chemical genetic modifier screen small molecule enhancers of rapamycin (SMERs) that augment (e.g., increase by at least 5%, 10%, 25%, 50%, 75%, 85%, 90%, or 95%) rapamycin's effect in the yeast Saccharomyces cerevisiae. Probing proteome chips with biotinylated SMERs will identify putative intracellular target proteins that modify a cellular effect of rapamycin. In one embodiment, rapamycin is added to the culture media of a yeast cell in the presence or the absence of a candidate compound. The yeast cell is maintained in culture and yeast cell proliferation is monitored (e.g., using optical density). A compound that reduces yeast cell proliferation in combination with rapamycin is identified as an SMER. Such compounds are likely to be useful for enhancing autophagy alone or in combination with rapamycin. If desired, the effect of the SMER on autophagy is assayed using any method described herein (e.g., increase in autophagy markers, increase in autophagic vesicle, enhanced degradation of a misfolded protein) or known in the art.

### Test Compounds and Extracts

In general, compounds capable of decreasing the amount of misfolded protein or increasing the selective degradation of such proteins in a cell are identified from large libraries of either natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in correcting a misfolded protein should be employed whenever possible.

When a crude extract is found to correct the conformation of a misfolded protein further fractionation of the positive lead extract is necessary to isolate chemical constituents responsible for the observed effect. Thus, the goal of the extraction, fractionation, and purification process is the careful characterization and identification of a chemical entity within the crude extract that increase the yield of a correctly folded protein. Methods of fractionation and purification of such heterogenous extracts are known in the art. If desired, compounds shown to be useful agents for the treatment of any pathology related to a misfolded protein or protein aggreagation are chemically modified according to methods known in the art.

### Combination Therapies

Compositions of the invention useful for the treatment of a PCD (e.g., retinitis pigmentosa, Huntington's disease, Parkinson's disease, Alzheimer's disease, nephrogenic diabetes insipidus, cancer, and prion-related disorders, such as Jacob-Creutzfeld disease) may, if desired, be administered in combination with any standard therapy known in the art. For retinitis pigmentosa, standard therapies include vitamin A supplements. In the case of Parkinson's disease, standard therapies include the administration of any one or more of the following dopamine receptor agonists levodopa/carbidopa, amantadine, bromocriptine, pergolide, apomorphine, benserazide, lysuride, mesulergine, lisuride, lergotrile, memantine, metergoline, piribedil, tyramine, tyrosine, phenylalanine, bromocriptine mesylate, pergolide mesylate; other standard therapies include antihistamines, antidepressants, dopamine agonists, monoamine oxidase inhibitors. For Huntington's disease, standard therapies include the administration of any one or more of the following haloperidol, phenothiazine, reserpine, tetrabenazine, amantadine, and co-Enzyme Q10. For Alzheimer's disease standard therapies include the administration of any one or more of the following: donepezil (Aricept), rivastigmine (Exelon), galantamine (Razadyne), and tacrine (Cognex). For nephrogenic diabetes insipidus standard therapies include the administration of any one or more of the following: chlorothiazide/hydrochlorothiazide, amiloride, and indomethacin. For cystic fibrosis, standard therapies include the administration of any one or more of mucus-thinning drugs (e.g., dornase alfa), bronchodilators (e.g., albuterol), and antibiotics for the treatment of infection. For cancer, standard therapies include the administration of any one or more of the following: abiraterone acetate, altretamine, anhydrovinblastine, auristatin, bexarotene, bicalutamide, BMS184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunin.

### Kits

The invention provides kits for the treatment or prevention of a PCD or symptoms thereof. In one embodiment, the kit includes a pharmaceutical pack comprising an effective amount of rapamycin or an analog thereof. In other embodiments, the kit includes rapamycin and 11-cis-retinal or 9-cis-retinal. In another embodiment, the kit includes an effective amount of a farnesyl transferase inhibitor (e.g., FTI-277). Preferably, the compositions are present in unit dosage form. In some embodiments, the kit comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired compositions of the invention or combinations thereof are provided together with instructions for administering them to a subject having or at risk of developing a PCD. The instructions will generally include information about the use of the compounds for the treatment or prevention of a PCD. In other embodiments, the instructions include at least one of the following: description of the compound or combination of compounds; dosage schedule and administration for treatment of a PCD or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The following examples are provided to illustrate the invention, not to limit it. Those skilled in the art will understand that the specific constructions provided below may be changed in numerous ways, consistent with the above described invention while retaining the critical properties of the compounds or combinations thereof.

### EXAMPLES

Retinitis pigmentosa (RP) is a PCD that comprises a heterogeneous group of inherited retinal disorders that lead to rod photoreceptor death. The death of photoreceptors results in night blindness and subsequent tunnel vision due to the progressive loss of peripheral vision in patients suffering from retinitis pigmentosa. Between 20-25% of patients with Autosomal Dominant Retinitis Pigmentosa (ADRP) have a mutation in the rhodopsin gene, the most common mutation being P23H. The P23H mutation results in a misfolded opsin protein that fails to associate with 11-cis-retinal. The misfolded P23H protein is retained within cells, where it forms aggregates (Saliba et. al. 2002. JCS 115: 2907-2918; Ming et. al. 2002. JBC 277: 34150-34160). This aggregation behavior classifies some RP mutations, including P23H, as protein conformational disorders (PCD).

Defects in the gene encoding cystic fibrosis transmembrane conductance regulator (CFTR) cause cystic fibrosis, which is another PCD. Cystic fibrosis is the most common lethal genetic disease in Caucasians, with about 30,000 cystic fibrosis patients in the United States. The CFTR forms a Cl⁻ channel that is an essential component of epithelial Cl⁻ transport systems in many organs, including the intestines, pancreas, lungs, sweat glands, and kidneys. In the Cl⁻ secretory intestinal epithelium, enters the cells through a Na⁺—K⁺-2Cl⁻ cotransporter in the basolateral membrane and exits through CFTR in the apical membrane; water follows osmotically. Defects in the gene encoding CFTR that reduce either its transport capacity or its level of cell surface expression cause cystic fibrosis. This defect in chloride transport leads to impaired clearance of airway secretions and a susceptibility to bacterial infection. Although cystic fibrosis is a multisystem disorder, respiratory failure remains the main cause of death.

While the following examples are directed to the use of specific mutant proteins for the identification of compounds that enhance the degradation of a mutant opsin or CFTR protein, the invention is not so limited. Compounds identified as useful for selectively enhancing the degradation (e.g., the autophagic degradation) of misfolded opsin or misfolded CFTR in a cell are useful for the treatment of retinitis pigmentosa or cystic fibrosis, respectively. Such compounds are likely to enhance the degradation of any misfolded protein, and are generally useful for the treatment of virtually any protein conformational disorder. Methods useful in carrying out the following experiments are described in Noorwez et al., Journal of Biological Chemistry 279:16278-16284 (2004), which is hereby incorporated by reference in its entirety.

### Example 1

### Autophagy Induction Results in the Degradation of Misfolded Proteins

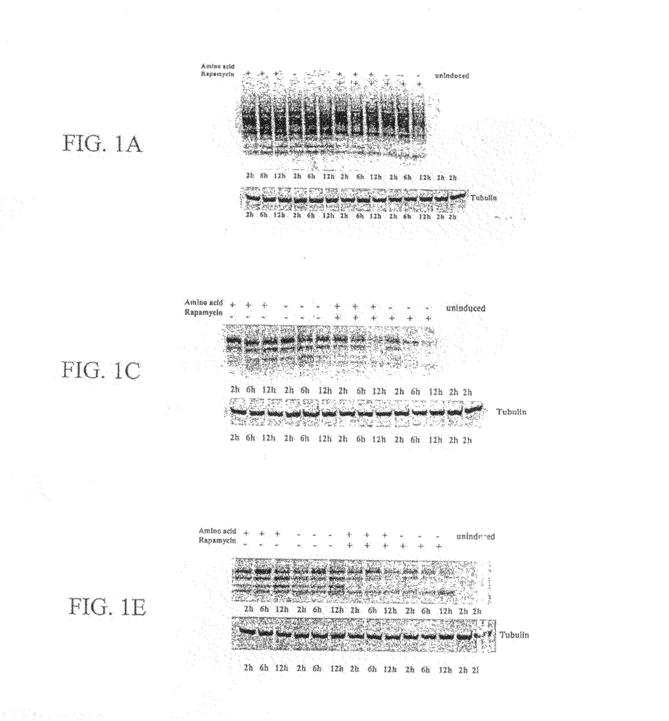

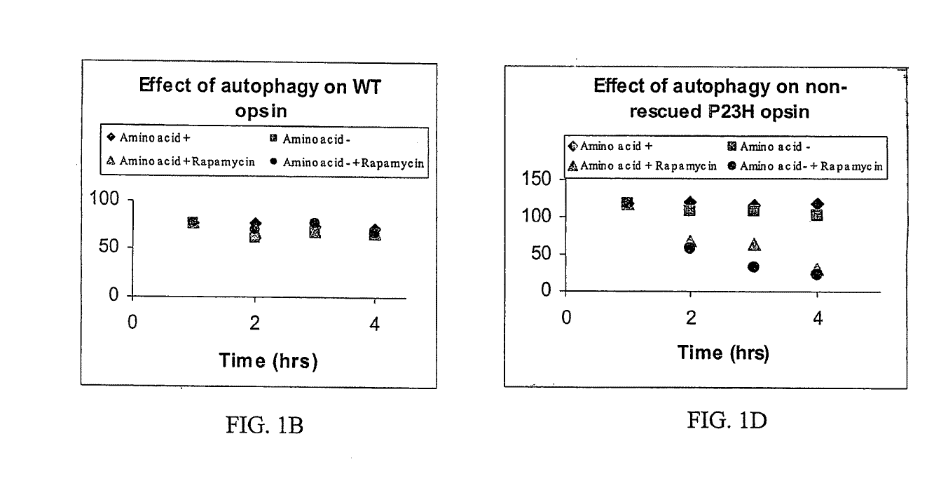

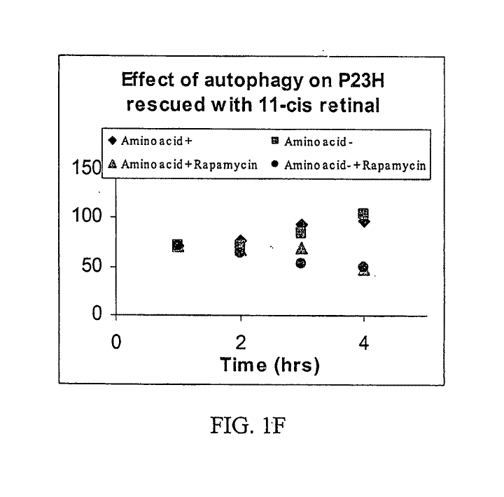

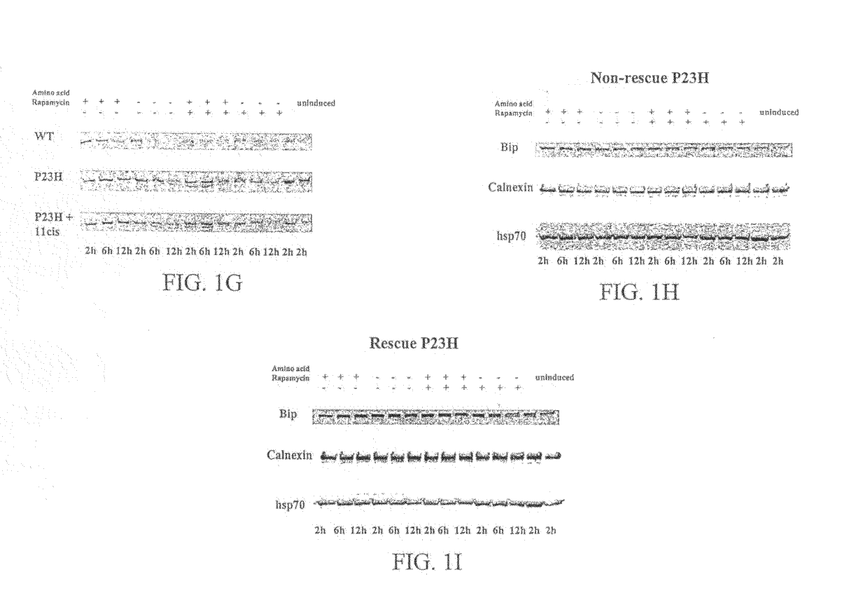

Using HEK293 tetracycline-inducible stable cell lines expressing either the P23H or wild-type opsin, macroautophagy was induced by amino acid depletion or by rapamycin exposure. FIG. 1A shows immunoblot degradation profiles of wild-type opsin when autophagy was induced by amino acid depletion alone (lanes 4-6) or by rapamycin (lanes 7-9), or by a combination of amino acid depletion and rapamycin exposure (lanes 10-12). The graph in FIG. 1B compares the relative amounts of wild-type opsin during the time course of the experiment and demonstrates that levels of wild-type opsin were essentially unchanged during the twelve hour course of autophagic induction. In contrast, the amount of P23H opsin decreased rapidly when autophagy was induced in cells by any of these methods (FIG. 1C). Degradation profiles of the P23H opsin (FIG. 1D) showed that when autophagy was induced by amino acid depletion, thirty-three percent of misfolded opsin was degraded within twelve hours (FIG. 1D, squares). When autophagy was induced by rapamycin, forty-six percent of protein was degraded within six hours of treatment (FIG. 1D, triangles). Degradation was further enhanced when amino acid depletion was combined with rapamycin treatment (FIG. 1D, circles), where almost fifty-two percent of misfolded opsin was degraded within two hours and eighty-one percent was degraded after twelve hours.

### Example 2

### Autophagy Specifically Degrades Misfolded Proteins

To determine whether this effect was modulated by proteasomal inhibition or whether it was mediated by autophagy alone, P23H degradation was examined in cells where autophagy was induced in the presence of a proteasomal inhibitor or an autophagic inhibitor. 3-methyladenine, an autophagy inhibitor, and MG132, a proteasomal inhibitor, were added to cell culture media at the time of autophagy induction. FIG. 1F shows that 3-MA inhibited P23H degradation in amino acid depleted cells. FIG. 1G shows that the proteasomal inhibitor MG132 had no effect on P23H opsin degradation (FIG. 1D). These studies indicate that autophagy specifically degrades misfolded P23H opsin.

### Example 3

### Rapamycin Enhances Autophagy of Misfolded Proteins

Previous studies showed that 11-cis retinal functions as a pharmacological chaperone that assists in the folding and stabilization of P23H opsin̂¹. Administration of 11-cis retinal allows most of the P23H protein pool to reach the cell surfacê¹, where it associates with 11-cis-retinal to form rhodopsin. When 11-cis retinal was administered at the time that autophagy was induced, levels of P23H degradation were not altered (FIG. 1E, lanes 4-6). Nevertheless, it is likely that administering 11-cis-retinal in combination with rapamycin for the treatment of an ocular PCD will have enhanced clinical efficacy because 11-cis-retinal will increase levels of correctly folded protein and rapamycin will enhance the degradation of any remaining misfolded protein.

Rhodopsin levels were similar in media supplemented with or without amino-acids (FIG. 1E, lanes 1-3, lanes 4-6). The degradation profile of P23H protein in cells treated with 11-cis retinal during amino acid depletion was compared to the degradation profile of cells expressing wild type opsin or P23H opsin-expressing cells that did not receive 11-cis retinal. In the presence of 11-cis-retinal, P23H protein degradation showed an intermediate degradation profile as compared to cells expressing wild-type opsin or expressing the P23H protein in the absence of 11-cis retinal.

Interestingly, treatment with rapamycin alone induced the rapid degradation of P23H rhodopsin regardless of whether it was administered alone or in combination with amino acid starvation of cells (FIG. 1E, lanes 7-9, 10-12). Degradation profiles P23H in the presence of 11-cis retinal were similar in cells cultured under normal conditions (FIG. 1F, diamonds) or in amino acid depleted media (FIG. 1F, squares). Enhanced degradation was observed when rapamycin was administered in the absence of 11-cis-retinal (FIG. 1F, triangles). Close to thirty-three percent of the P23H protein was degraded within twelve hours. When rapamycin was administered to amino-acid starved cells no further increase in rhodopsin degradation was observed. In fact, administration of rapamycin to amino acid depleted cells was similar to the effect of rapamycin treatment alone. Almost thirty-one percent of P23H protein was degraded within twelve hours (FIG. 1F, circles). Thus, autophagy induced by rapamycin selectively increased P23H protein degradation in the presence of 11-cis retinal.

### Example 4

### Autophagy Induction Induced Characteristic Changes in mTOR Phosphorylation

mTOR is the mammalian target of rapamycin, and mTOR levels are characteristically reduced in autophagic cells. To confirm that autophagy was induced using the methods described above, mTOR phosphorylation was characterized in amino acid depleted cells and in cells that received rapamycin. As expected, a decrease in the amount of phosphorylated mTOR was observed following autophagy induction in both amino-acid depleted and rapamycin treated cells. Augophagic induction was characterized in cells expressing wild-type or P23H opsin, as well as in cells that were also administered 11-cis-retinal. This increase in phosphorylation was specific to mTor, since cellular levels of Bip and calnexin, chaperones upregulated by unfolded protein response (UPR), and Hsp70, a cytoplasmic chaperone upregulated by heat shock response (HSR), were unchanged under autophagic conditions. The amounts of these chaperones remained constant over time (FIG. 1H, 1I) following autophagy induced by amino-acid starvation or rapamycin treatment.

To determine whether autophagy provides a general mechanism for the degradation of misfolded proteins, the autophagic degradation of a mutant cystic fibrosis transmembrane conductance regulator (CFTR) protein was characterized. CFTR is a cAMP-activated chloride channel expressed at the apical membrane of epithelial cells. Like P23H opsin, CFTR is a polytopic membrane protein. Such polytopic proteins have many alpha helical transmembrane segments. Mutations in CFTR affect its ability to be made, processed, and trafficked to the plasma membrane, where its function is required.

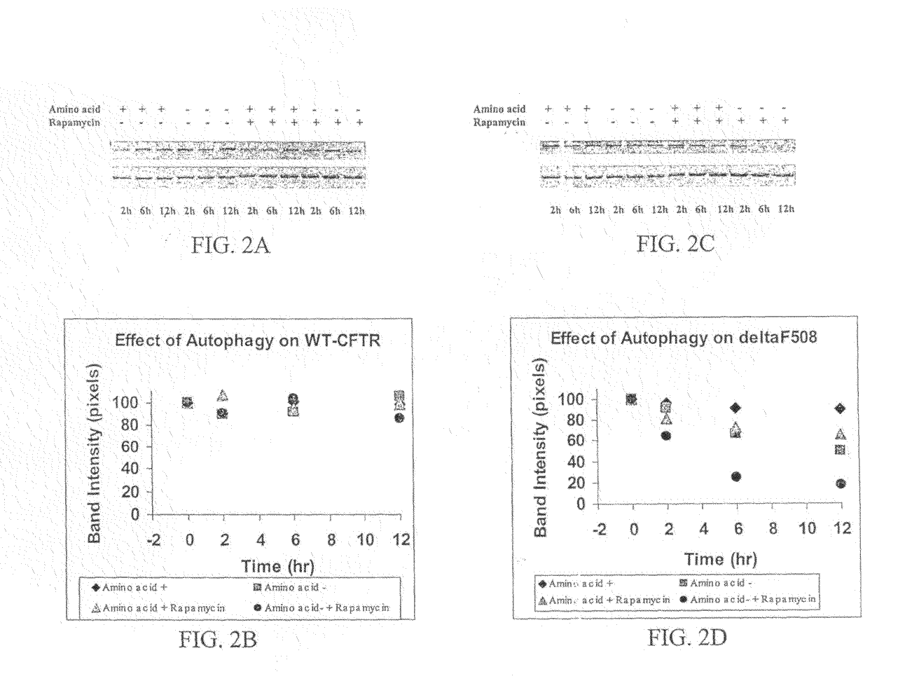

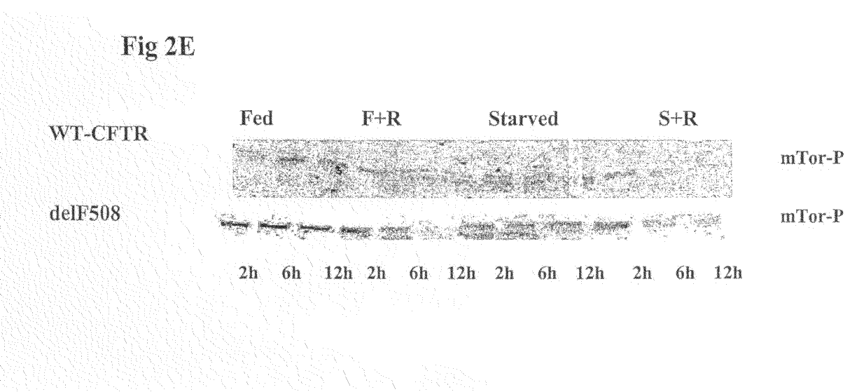

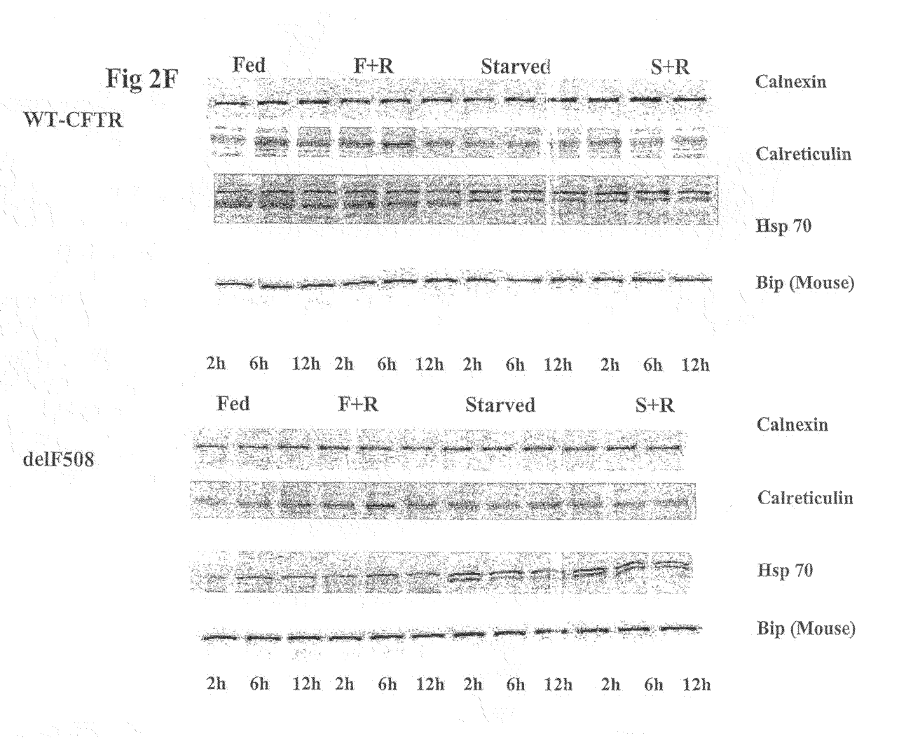

Wild-type CFTR (FIG. 2A) and mutant CFTR ΔF508 (FIG. 2C) proteins were expressed in BHK stable cell lines. Autophagy was induced in cells expressing the wild-type or mutant CFTR proteins by amino-acid depletion, rapamycin treatment, or a combination of rapamycin treatment and amino acid depletion (FIG. 2A). While autophagic induction did not affect wild-type CFTR protein levels (FIG. 2B), the ΔF508 protein (FIG. 2D) underwent rapid degradation in response to autophagic induction by either amino-acid depletion, rapamycin treatment, or both (FIG. 2C). Degradation profiles indicated that amino-acid starvation degraded about thirty-four percent of the ΔF508 protein within twelve hours (FIG. 2D, triangles), while rapamycin treatment caused fifty percent of protein to be degraded after twelve hours (FIG. 2D, squares). Degradation was greater when amino acid starvation was combined with rapamycin treatment. Nearly seventy-five percent of the mutant protein was degraded within six hours and eighty percent was degraded after twelve hours of treatment (FIG. 2D, circles). These observations indicate that autophagy selectively degrades misfolded ΔF508 protein. MTor phosphorylation was reduced following autophagic induction in cells expressing either wild-type or mutant CFTR protein. No difference in the levels of chaperones, Bip, calnexin and hsp70 were observed. FIGS. 2E and 2F are immunoblots showing mTor dephosphorylation (FIG. 2E) and calnexin, calreticulin, Hsp70, or Bip protein expression in cells cultured in normal media or amino acid depleted media in the presence or absence of rapamycin.

### Example 5

### Autophagic Markers Colocalize with Misfolded Proteins Following Autophagy Induction

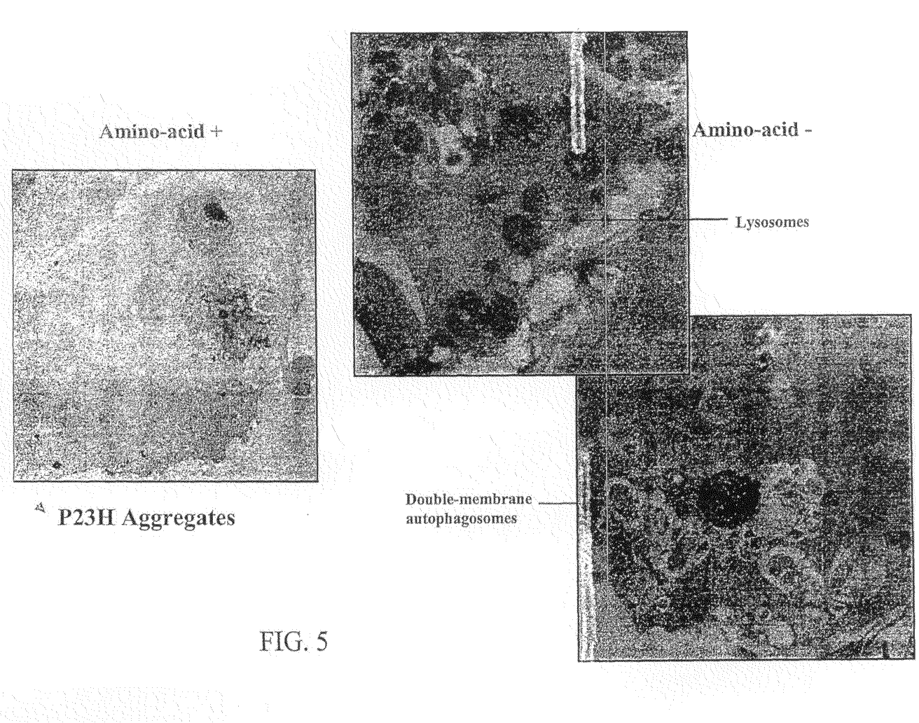

Autophagy induction can be monitored by identifying the presence of autophagic vacuoles (AVs) in cells using electron microscopy. P23H cells in normal media contain some AVs, but the number of such vacuoles is significantly increased when the cells are incubated in amino acid depleted media (FIG. 5).

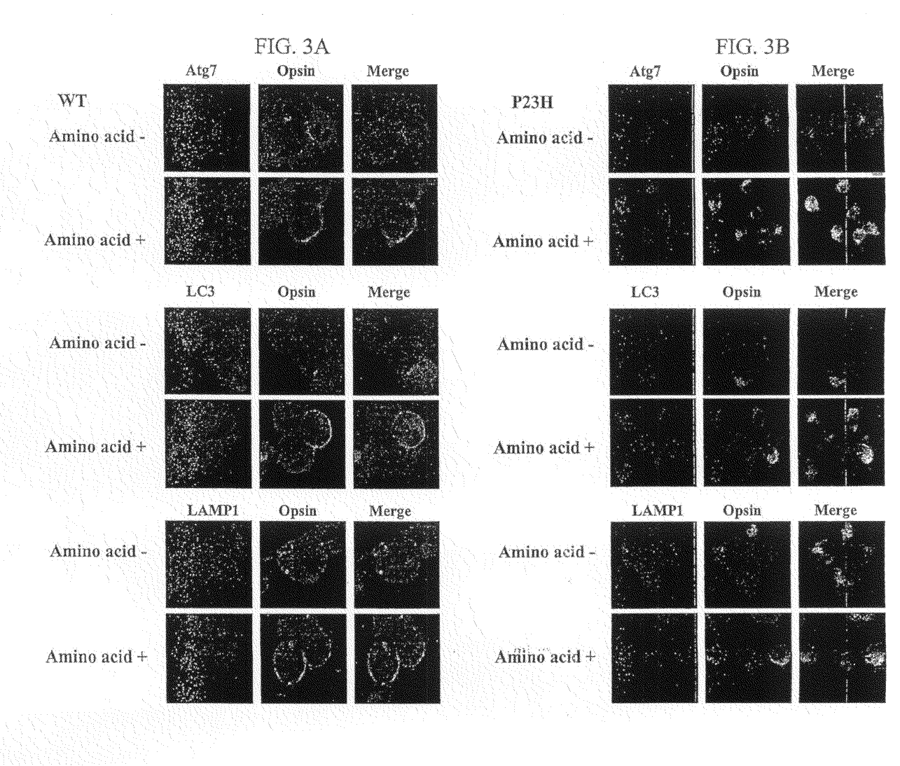

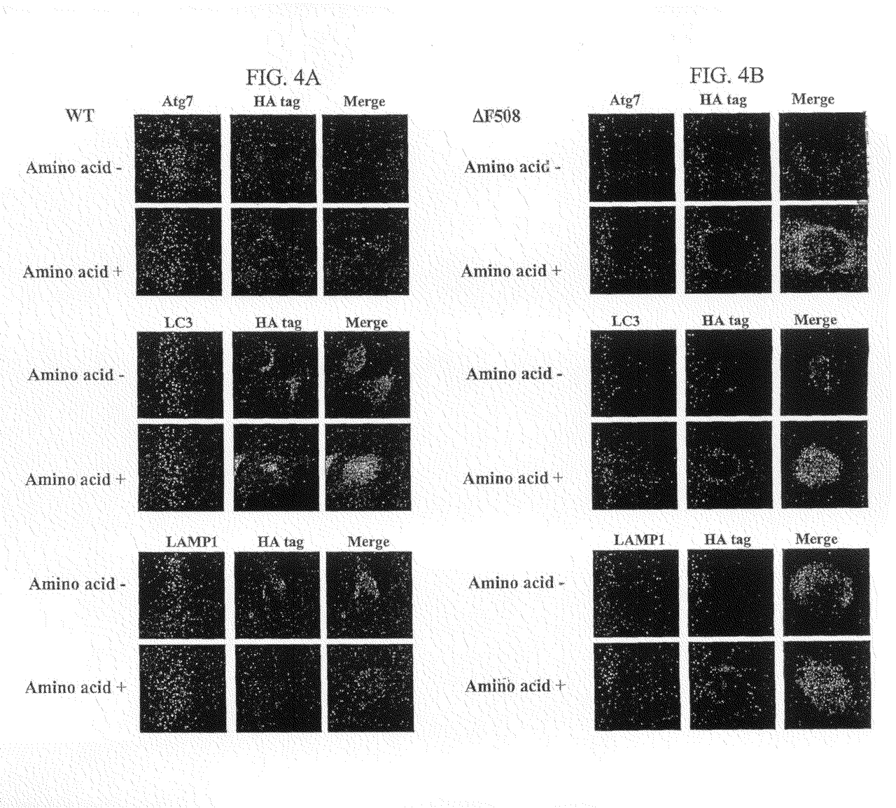

To determine whether misfolded proteins co-localize with autophagosome markers, cells expressing wild-type (FIG. 3A) or mutant P23H opsin (FIG. 3B) were stained for Atg7, LC3, and Lamp 1 autophagic markers. Cells were incubated with opsin specific and autophagosome-marker specific antibodies following autophagy induction. The wild-type protein failed to colocalize with any autophagosome marker (FIG. 3A). The wild-type opsin protein was present at the cell membrane, while the P231-1 formed intracellular aggregates. The same autophagic markers were examined in BHK cells expressing wild-type (FIG. 4A) or mutant ΔF508 CFTR protein (FIG. 4B). In these cells the ΔF508 CFTR protein did not form visible aggregates. The wild type CFTR protein was observed at the cell membrane as well as intracellularly (FIG. 4A), while the ΔF508 was retained in the ER (FIG. 4B). FIGS. 5A-5C are electron micrographs showing P23H aggregates, lysosomes, and autophagic cells.

Immunoflourescence staining with Atg 7 showed punctate staining that colocalized with the both mutant proteins. Colocalization of Atg 7 with P23H is shown in FIG. 3B and co-localization with ΔF508 is shown in FIG. 4B. Atg8 staining also co-localized with both mutant proteins. Atg8 showed distinct punctate staining that colocalized with misfolded protein aggregates of P23H (FIG. 3B). Atg8 staining also colocalized with the ΔF508 protein that was retained in the ER (FIG. 4B). These observations further support a role for autophagy in the degradation of mutant polytopic proteins, such as P32H and ΔF508.

In summary, the autophagic pathway specifically degraded misfolded polytopic proteins, such as P23H and ΔF508 while having very little effect on wild-type proteins. This result was independent of the cell line used to express the mutant proteins, since wild-type and mutant opsin proteins were expressed in a human embryonic kidney cell line while wild-type and mutant CFTR proteins were expressed in baby hamster kidney cell lines.

Autophagosomes were visualized using electron microscopy. Increased numbers of double membrane autophagic vacuoles were observed in cells that expressed P23H opsin. Following autophagic induction these cells contained large aggregates or small disintegrated aggregates of P23H opsin. Dark acid phosphatase stained lysosomes were also found observed in association with the AV's and aggregates. Without wishing to be tied to one particular theory, this could indicate a role for the lysosomal pathway in the degradation of misfolded opsin.

Autophagosome markers colocalized with misfolded P23H opsin and ΔF508 proteins. Atg7 is a key autophagic gene encoding a protein resembling E1 ubiquitin-activating enzyme required for formation of AVs. Atg7 promotes the conjugation of Atg8, a microtubule associated protein light chain 3, to the lipids that form the sequestering membranes of the AVs and enhances their formation. Atg8 exists at the membranes of both the early and late autophagosomes. Distinct punctuate staining of Atg7 and Atg8 colocalizing with P23H and ΔF508 proteins suggests a role for AVs in the degradation of misfolded proteins.

### Example 6

### Rapamycin Treatment Enhances Retinal Function in Retinitis Pigmentosa

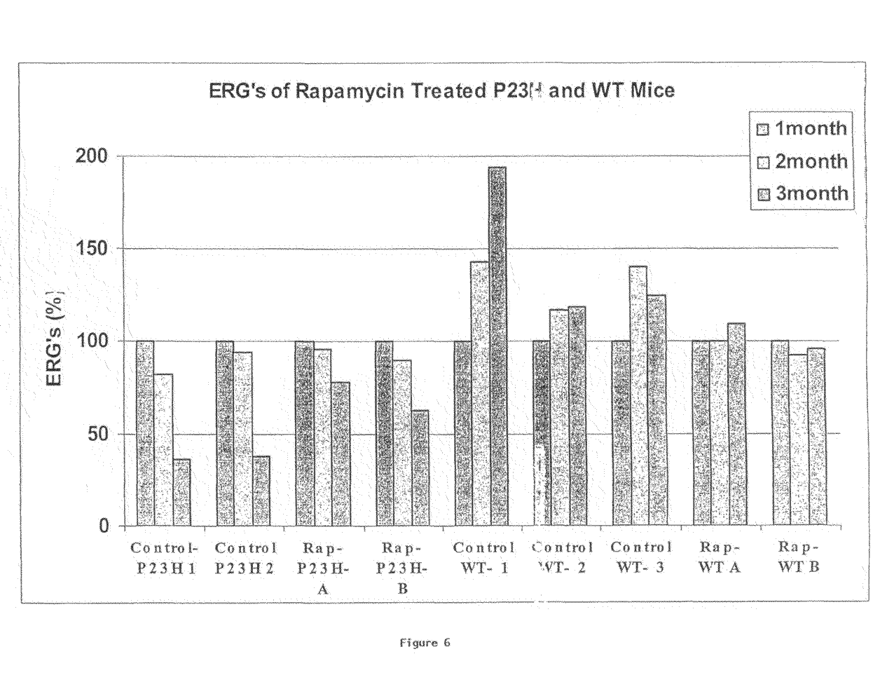

Transgenic mice expressing mutant mouse opsin having a P23H mutation undergo a rapid progressive photoreceptor degeneration that resembles the pathophysiological changes observed in retinitis pigmentosa patients. In vivo studies with P23H heterozygous mutant mice showed that rapamycin treatment rescued retinal function over a course of three months (FIG. 6).

### Example 7

### Rapamycin Enhances Retinal Function in Macular Degeneration

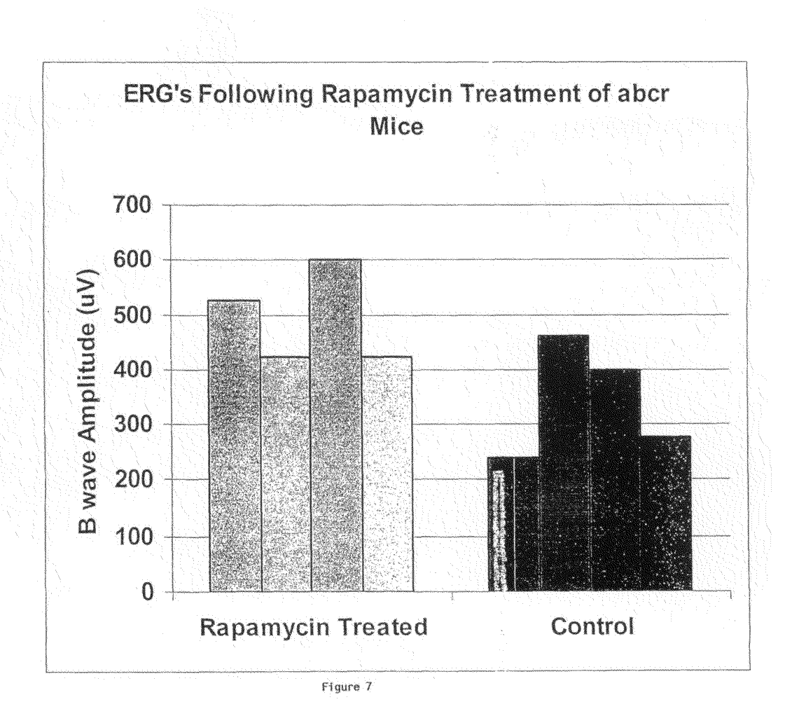

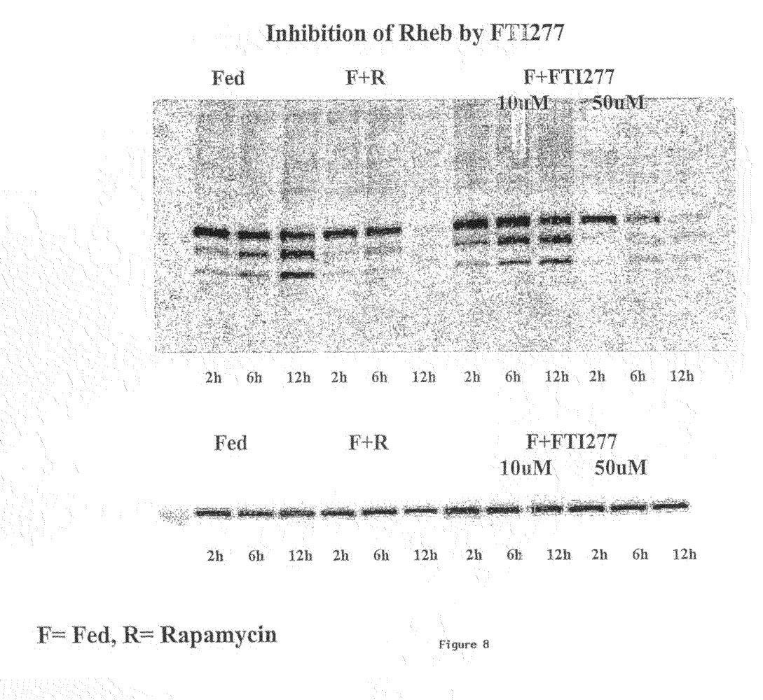

The bis-retinoid fluorophores that accumulate in retinal pigment epithelial (RPE) cells as lipofuscin constituents are considered to be responsible for the loss of RPE cells in recessive Stargardt disease, an early onset form of macular degeneration, and may also be involved in the etiology of age-related macular degeneration. In vivo studies in a mouse model of macular degeneration show that rapamycin treatment rescues retinal function in Abcr heterozygous mutant mice having one defective copy of the ABCR gene, which is associated with Stargardt disease (FIG. 7). The ABCR gene encodes rim protein (RmP), an ATP-binding-cassette transporter expressed in the rims of photoreceptor outer-segment discs.

### Example 8

### FTI277 Induced the Rapid Degradation of P23H Rhodopsin

Treatment with the farnesyl protein transferase inhibitor, FTI277, methyl {N-[2-phenyl-4-N[2(R)-amino-3-mercaptopropylamino]benzoyl]}-methionate (Calbiochem) induced the rapid degradation of P23H rhodopsin just as rapamycin did (FIG. 8). It is likely that FTI277 enhances autophagy just as rapamycin does, and that FTI277 is useful for the treatment of protein conformation diseases.

### Example 9

### FTI-277 Stimulates the Degradation of P23H Opsin

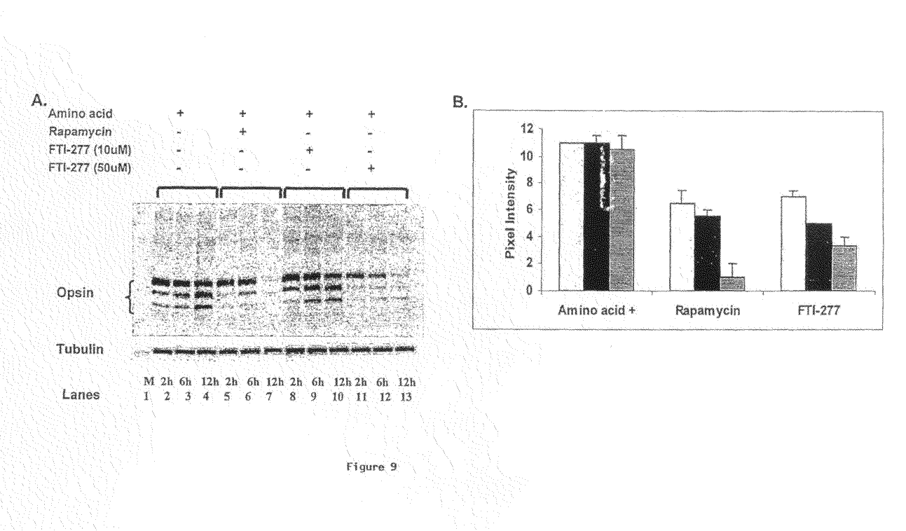

To study the effect of FTI-277 on the levels of mutant opsin, HEK293 cells expressing P23H opsin were incubated with different concentrations (1, 5, 10 and 50 μM) of FTI-277. A time-dependent degradation of P23H opsin at 50 μM was observed. No effect of FTI-277 was detected at 10 μM (FIG. 9A). When compared to rapamycin, 70% of P23H opsin was lost after 12 hours of rapamycin treatment, while FTI-277 resulted in a 50% loss of P23H opsin during this time period (FIG. 9B).

### Example 10

### FTI-277 Does Not Induce UPR/HSR

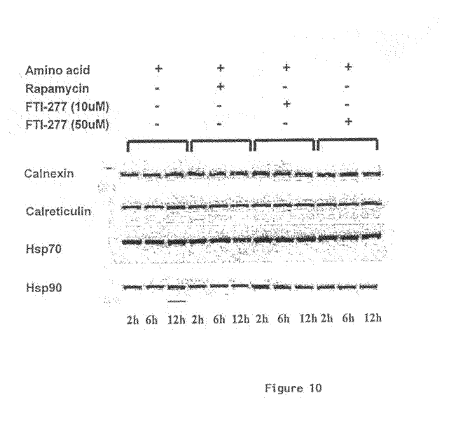

Following treatment with FTI-277, differences in the levels of calnexin and calreticulin, endoplasmireticulum chaperones involved with the unfolded protein response (UPR), or Hsp70 and Hsp90 levels, cytoplasmic chaperones associated with heat shock response (HSR) were analysed. FTI-277 did not affect the levels of either of the two responses, suggesting that FTI-277 induced degradation of P23H opsin is exclusive of both UPR and HSR (FIG. 10).

### Example 11

### FTI-277 Treatment Blocks mTOR/S6K Signaling

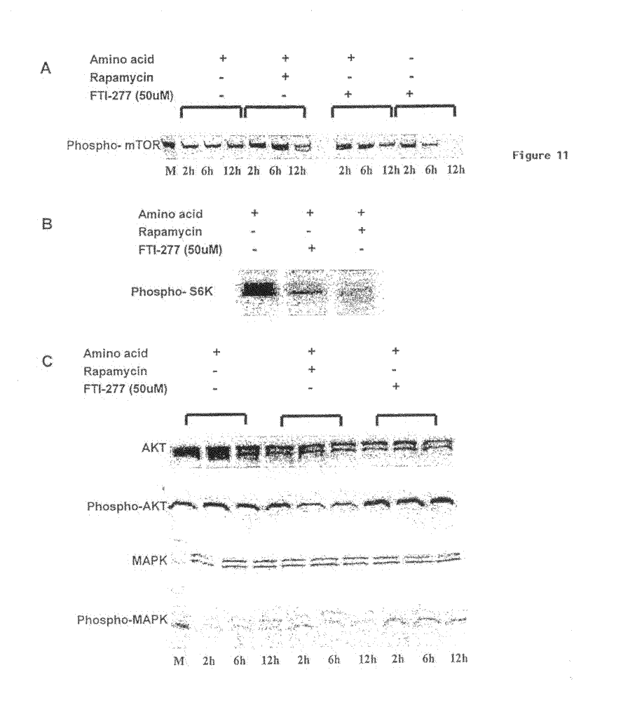

FTI-277 effectively inhibited the phosphorylation of mTor and S6 kinase as predicted if this drug was suppressing Rheb activity (FIG. 11A, B). Phosphorylated mTor and S6 kinase were observed in amino acid and serum fed cells. As with rapamycin treatment, FTI-277 induced the dephosphorylation of mTOR, which was further enhanced in combination with amino acid and serum starvation (FIG. 11A). Similarly, the phosphorylation of S6 kinase was dramatically reduced in cells treated with FTI-277 or rapamycin (FIG. 11B). In contrast, stimulation of Akt phosphorylation in HEK293 cells was unaffected by FTI-277 treatment suggesting that Ras activity was unaffected, although a slight increase in MAPK phosphorylation was observed similar to Basso et al., J. Biol. Chem. 280, 31101-31108, 2005 (FIG. 11C).

### Example 12

### Colocalization of P23H Opsin with Atg7 and Atg8 upon FTI-277 Treatment

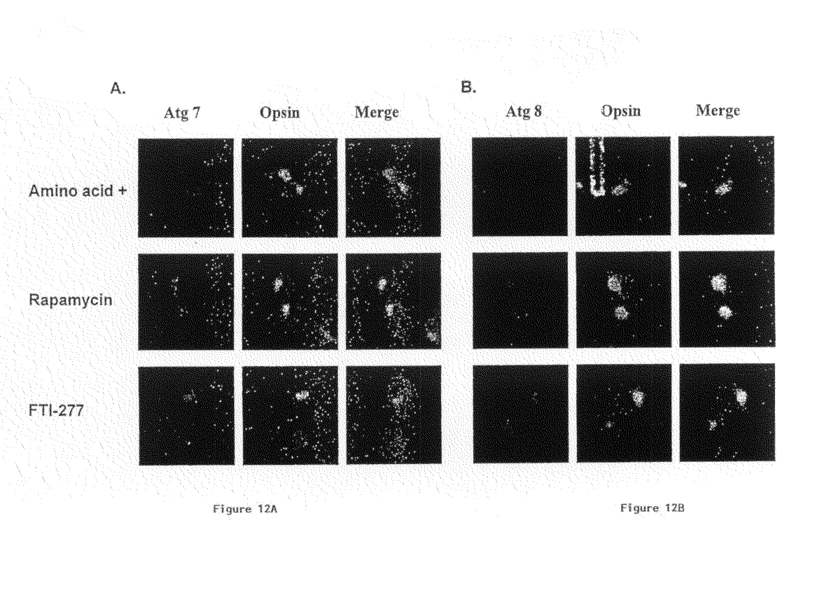

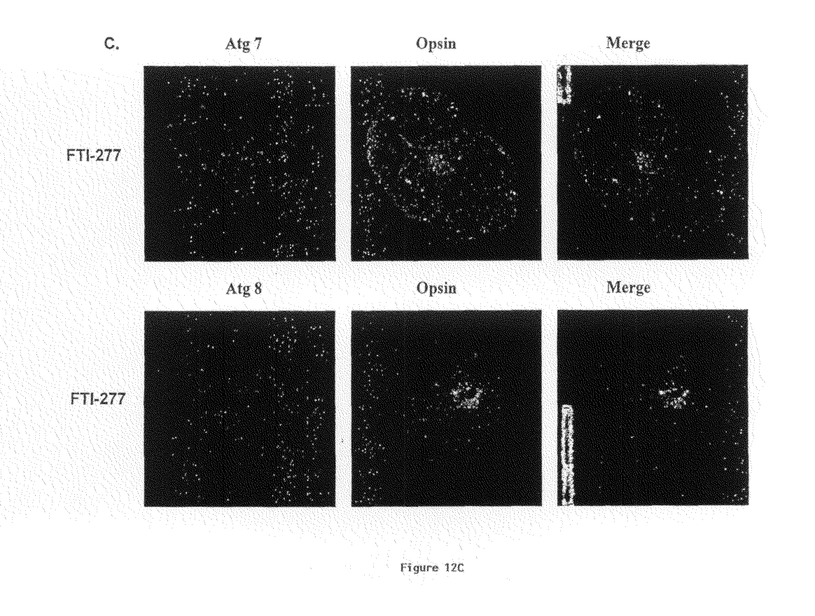

Given that the rapamycin-induced degradation of P23H opsin was mediated by autophagy, it is likely that FTI-277 degradation proceeds through autophagy, as well. The relationship of P23H opsin aggregates with known autophagosome markers, Atg7 and Atg8, was analysed by immunofluorescence microscopy. These markers do not normally localize with P23H opsin in untreated cells grown in complete medium (FIGS. 12A and 12B). A dramatic increase in the colocalization of both the markers with P23H opsin upon FTI-277 treatment was observed (FIG. 12A and FIG. 12B). The location of Atg7 and Atg8 with respect to P23H opsin aggregates was better observed by confocal microscopy (FIG. 12C). Atg7 dots appear clustered with the P23H opsin aggregates, colocalizing with P23H opsin. Similarly, there is enhanced colocalization of some Atg8 dots with P23H opsin aggregates, while the rest of the dots were situated around the aggregate. The presence of Atg7 and Atg8 proteins within and around the aggregates supports a role of autophagy in degradation of P23H opsin.

### Example 13

### The Induction of Autophagy by FTI-277

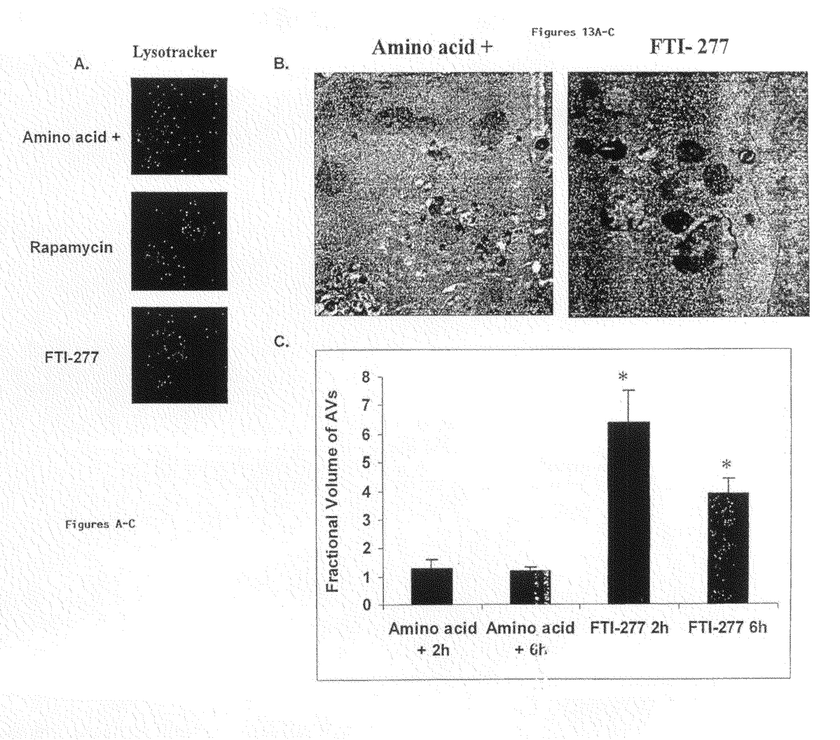

These results indicate that FTI-277 activates autophagy. Therefore, the autophagic response to FTI-277 was analysed using lysotracker to visualize the number and size of lysosomal vacuoles in the cell. An increase in lysosomal numbers and size was observed when cells were treated with rapamycin or FTI-277 (FIG. 13A). Next, electron microscopy was utilized to assess the autophagic responses in untreated and FTI-277 treated HEK293 cells expressing P23H opsin. As seen by lysostraker, FTI-277 treated cells contained many large autophagic vacuoles containing lysosomal acid phosphatase (FIG. 13B). Furthermore, some of these vacuoles appeared to be in the process of engulfing large cytoplasmic aggregates (FIG. 13B). Upon morphometric quantification, a 4 to 6-fold increase in the fractional volume of AVs in FTI-277 treated cells was found compared to untreated cells (FIG. 13C). These data suggest that FTI-277 promoted the autophagic response in HEK293 cells.

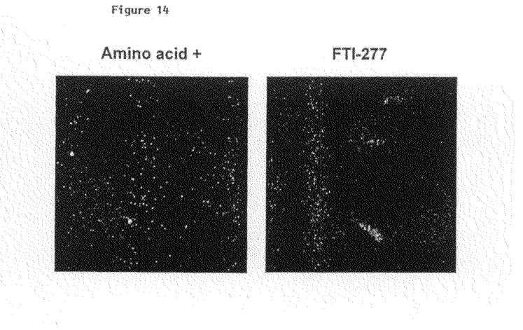

The effects of FTI-277 on HuH7 hepatoma cells stably expressing GFP-LC3, a marker of autophagy, was also investigated. In fed cells, GFP-LC3 was predominantly found diffusely throughout the cytoplasm. When these cells were treated with FTI-277, GFP-LC3 localized to numerous structures consistent with its association with autophagic vacuoles and the onset of autophagy (FIG. 14). These studies demonstrate that autophagy is dramatically upregulated in cells treated with FTI-277.

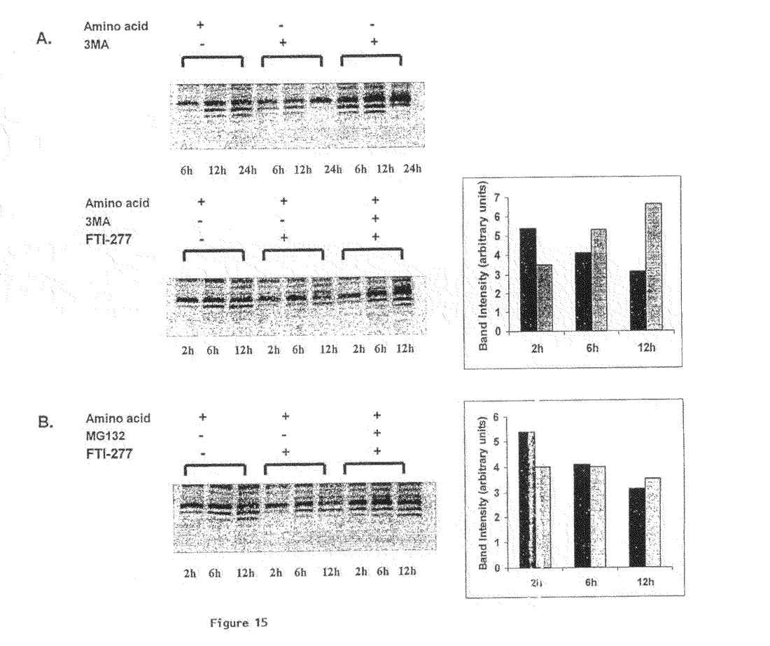

The induction of autophagy in P23H opsin expressing HEK293 cells was examined using a PI3-kinase inhibitor, 3-methyladenine (3MA), which blocks autophagy. 3MA treatment in the presence of FTI-277 prevented degradation of opsin. This effect was not observed when the cells were treated with FTI-277 alone (FIG. 15A). To further confirm that P23H opsin degradation was autophagic, the proteasomal inhibitor, MG132 was used. P23H opsin degradation had similar kinetics in the presence of FTI-277 and MG132 as with FTI-277 alone, suggesting that the role of proteasomal degradation in this pathway was limited (FIG. 15B).

Farnesyl transferase inhibitors (FTIs) were originally designed to block the action of Ras oncoproteins. The activity of Ras depends on farnesylation, a posttranslational modification that links a farnesyl isoprenoid membrane anchor to the protein. Farnesyl transferases catalyze the transfer of a 15-carbon isoprenyl lipid from farnesyl diphosphate onto a cysteine residue of various protein substrates. Farnesyl transferases recognize the carboxyl terminal CAAX box of the substrate.

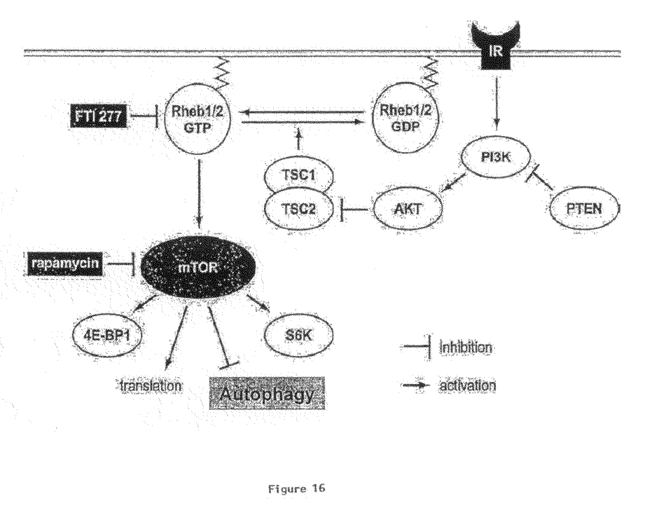

Rheb is a guanine nucleotide binding protein and a GTPase. Rheb proteins contain G1-G5 boxes which are short stretches of sequences involved in the recognition and hydrolysis of GTP (Bourne et al., (1990) Nature 348, 125-132). Also, Rheb proteins end with a CAAX (CSVM) motif that is required for farnesylation. In mammalian cells, the ability of Rheb to activate S6K has been established. This function is dependent on farnesylation, since Rheb mutants lacking the CAAX motif cannot activate S6K (Castro et al., J. Biol. Chem. 278, 32493-32496, 2003; Tee et al., Curr. Biol. 13, 1259-1268, 2003). Moreover, it has been well established that FTIs, like FTI-277, completely block the prenylation of Rheb (Basso et al., J. Biol. Chem. 280, 31101-31108, 2005). Rheb does not undergo geranylgeranylation. There are other targets of FTIs besides Rheb. Studies show that proteins like K-Ras4B show resistance to FTIs because they undergo geranylgeranylation when farnesylation is inhibited [(22, 23). These studies suggest that Rheb is a more specific target of FTIs than other proteins that are subject to farnesylation. In addition, Akt phosphorylation is not affected by FTI-277, suggesting that FTI-277 dose not affect Ras activity. Rheb is a component of the insulin/TOR/S6K signaling pathway (Castro et al., J. Biol. Chem. 278, 39921-39930, 2003; Tabancay et al., J. Biol. Chem. 278, 39921-39930, 2003; Tee et al., Curr. Biol. 13, 1259-1268 24, 2003; Inoki et al., Genes Dev. 17, 1829-1834, 2003; Garami et al., Mol. Cell. 11, 1457-1466, 2003). As reported herein, dephosphorylation of mTOR and S6K with FTI-277, consistent with the inhibition of Rheb, was observed. A similar decrease in phosphorylation of mTOR and S6K are observed when autophagy is induced in cells by use of rapamycin. Besides inhibiting mTOR, these results indicate that autophagy can be induced in cells by FTI-277 treatment, which blocks Rheb further upstream of mTOR (FIG. 16).

Treatment of P23H opsin expressing cells with FTI-277 at 50 μM induced degradation of mutant opsin as did rapamycin treatment. Immunofluorescence studies using antibodies to autophagosome markers Atg7 and Atg8 confirmed the induction of autophagy in these cells. Atg7 is a key autophagic gene encoding a protein resembling E1 ubiquitin-activating enzyme required for formation of AVs (Tanida et al., (2001) J. Biol. Chem. 276, 1701-1706. Atg7 promotes the conjugation of Atg8, a microtubule-associated protein light chain 3, to the lipids that form the sequestering membranes of the AVs (Ohsumi et al., Nat. Rev. Mol. Cell. Biol. 2, 211-216, 2001; Kabeya et al., J. Cell Sci. 117, 2805-2812, 2004). Both markers colocalized with P23H opsin. Furthermore, the ultrastructure studies performed using electron microscopy revealed the increased expression of AVs in cells when treated with FTI-277. In some micrographs, AVs engulfing the cytoplasmic aggregates was also observed. Furthermore, the morphometric analysis showed a 6-fold increase in the fractional volume of AVs in cells treated with FTI-277, thus firmly establishing the induction of autophagy in these cells.

In summary, these data suggest that the autophagic pathway can be stimulated not only by blocking mTOR with rapamycin, but also by modulating components upstream of mTOR using small molecule farnesyltransferase inhibitors, such as FTI-277. Like other FTIs, FTI-277 reduces the farnesylation of Rheb, thereby inactivating this G-protein. These studies open the possibility of using FTIs for the treatment of various protein conformational disorders (PCDs), since autophagy is involved in degrading mutant, aggregated proteins implicated in various neurodegenerative diseases including Parkinson's (Cuervo et al., Science 305, 1292-1295, 2004), Huntington's disease (Ravikumar et al. Nat. Genet. 36, 585-595, 2004). Stimulating autophagy in Huntington's disease (Ravikumar et al. Nat. Genet. 36, 585-595, 2004), both in cell culture and mouse model, and Autosomal Dominant Retinitis Pigmentosa (ADRP) leads to loss of accumulated aggregates. Also, the current use of FTIs in clinical trials (phase II and III) for cancer further assures the lack of toxicity of this compound in both humans and tested animals. FTI drugs may provide a therapeutic alternative to rapamycin especially in enhancing the removal of protein aggregates by autophagy.

The experiments described above were carried out using the following materials and methods.

### Mammalian Cell Cultures

Wild-type and P23H opsin were expressed in HEK293 tetracycline-inducible stable cell lines. The cells were grown in Dulbecco's modified Eagle's medium containing high glucose (Invitrogen, San Diego, Calif.) supplemented with 10% heat inactivated fetal bovine serum (Sigma) with antibiotic-antimycotic solution (Invitrogen, San Diego, Calif.), blasticidin (Cayla, Toulouse, France), zeocin (Invitrogen, San Diego, Calif.) at 37 C in the presence of 5.0% CO₂. Opsin synthesis in cells was induced by addition of tetracycline (1 μg/ml). Baby hamster kidney (BHK) cell lines stably expressing the wild type and ΔF508 CFTR variant with a C-terminal HA epitope (CFTR-HA) (See Sharma et al., J. Cell Biol. 2004 164(6):923-33). The cells were grown in DMEM/F12 (Invitrogen, San Diego, Calif.) 1:1 ratio with 10% FBS at 37° C. in the presence of 5.0% CO₂.

HuH7 hepatoma cells were stably transfected with pGFP-LC3 (Ogawa et al., Science 307(5710): 727-731, 2005) using a lipid optimization kit, the PerFect lipid (pFx-3), purchased from Invitrogen (San Diego, Calif.). PFX-3 (Invitrogen) according to manufacturer's protocols. Those colonies that grew in the presence of 0.5 mg/ml G418 were then isolated, amplified, and screened for GFP-LC3 expression by fluorescence microscopy and Western blotting.

### Induction of Autophagy

Autophagy was induced in cells by incubating them in amino-acid depleted medium or treating them with rapamycin (50 mM), or both. Cells were incubated under autophagy inducing conditions for two, six, or twelve hours. At the indicated time point the cells were lysed in 1% n-dodecyl-β-maltoside (DM) (Anatrace, Maumee, Ohio) in the presence of protease inhibitors (complete protease inhibitor mixture tablets (Roche Molecular Biochemicals, Mannheim, Germany) for 1 hour at 4° C. The cells were centrifuged at 36,000 rpm in a Beckman ultracentrifuge for 30 minutes at 4 C. The lysate was collected and immunoblotting was performed.

### SDS Gel Electrophoresis and Immunoblotting

Cell lysates were electrophoresed on 10% SDS polyacrylamide gels and transferred onto Immobilon-NC (Millipore, Billerica, Mass.) nitrocellulose membranes. The membranes were incubated at room temperature for 1 hour with a commercially available blocking buffer (Li-Cor, Lincoln, Nebr.) diluted 1:1 in PBST (PBS with 0.1% triton X-100, pH 7.4), followed by a 1 hour incubation with the indicated primary antibody. The blots were washed three times for 5 minutes each in PBST, and incubated for 1 hour with a true near infrared dye, IRDye800-conjugated secondary antibody (Rockland Immunochemicals Inc., Gilbertsville, Pa.). Finally the membranes were again washed three times with PBST and scanned in an Odyssey infrared scanner (Li-Cor, Lincoln, Nebr.). Quantitations on immunoblots were performed using Licor software. Primary antibodies included antibodies to opsin, HA-tag (Covance Princeton, N.J.), mTOR, phosphorylated mTOR (Upstate Charlottesville, Va.), calnexin, hsp70 (Stressgen, Victoria, BC, CA), Bip (BD PharMingen, San Diego, Calif.) to tubulin (Sigma Chemical, St. Louis, Mo.), 1D4 (University of British Columbia), Akt, phospho-Akt, S6K, phospho-S6K, MAPK and phosphor-MAPK (Cell Signaling Technology, Beverly, Mass.).

### Immunoflourescence

Cells were grown on glass coverslips and fixed in 4% paraformaldehyde. Following quenching with 50 mM NH₄Cl, cells were washed with PBS and incubated for 1 hour with the indicated primary antibody at room temperature. Cells were washed five times in PBS and incubated with a secondary antibody (TRITC- and FITC-conjugate) for 1 hour. The cells were washed again and mounted with Vectashield containing DAPI. Primary antibodies included antibodies to LC3, LAMP-1, Atg7 (Dr Dunn), opsin, Atg7²⁰, Atg8 and HA-tag. The cells were then observed using a Zeiss Axiophot microscope.

Staining with lysotracker (Molecular Probes) was also performed on live cells at 37° C. Confocal imaging was performed using the Leica TCS SP2 AOBS Spectral Confocal Microscope at 63× magnification.

### Mouse Models

abcr+/− mice are described by Mata et al., Investigative Opthalmology and Visual Science. 2001; 42:1685-1690.

Mice expressing the P23H opsin protein are described by Liu et al., Journal of Cell Science 110, 2589-2597 (1997).

### In Vivo Rapamycin Treatment

abcr+/− mice were treated with 20 mg/kg of rapamycin once per week beginning when the mice were four months old. The rapamycin was administered by intraperitoneal injection.

Heterozygous P23H transgenic mice were treated once per week with rapamycin beginning when the mice were twenty-one days old.

### Electroretinography

The mice are dark adapted twenty-four hours prior to ERG. A mix of ketamine and xylazine was used to anesthestize mice. Dosage was determined by weight. Mouse eyes were numbed with proparacaine drops and dilated with Ak-dilate. The mouse was then placed on the machine (UTAS-E 2000) a ground electrode was inserted into the hind limb, another electrode was inserted into the neck, and a pair of electrode was used to record the ERG from each eye. After getting a baseline reading, the ERG was measured at 20, 10 and 0 dB. Monthly ERG's were measured and the B-wave amplitude was determined.

### FTI-277 Treatment

FTI-277 (Calbiochem) was used at 50 μM. Following tetracycline wash off, cells were treated for 0, 2, 6 and 12 hours then lysed in phosphate buffer containing 1% n-dodecyl-β-maltoside (DM) (Anatrace) in the presence of protease inhibitors (complete protease inhibitor mixture tablets; Roche Molecular Biochemicals) for 1 hour at 4° C. As a positive control for autophagy, cells were treated with rapamycin (50 nM), following tetracycline wash off. The lysates were centrifuged at 36,000 rpm in a Beckman ultracentrifuge for 10 min at 4C. The supernatant was collected and immunoblotting was performed. 3-methyladenine (3MA), which blocks autophagy (10 mM) (Sigma Chemical (St. Louis, Mo.)) and MG132 (25 μM) (Sigma Chemical (St. Louis, Mo.)) were also used.

### Electron Microscopy

P23H opsin-expressing cells were grown on ACLAR sheets in a 24-well plate. Opsin production was induced by the addition of tetracycline for 48 hours and cells were treated with FTI-277 for 6 hours after tetracycline removal. Following a wash with PBS, cells were fixed with 2% paraformaldehyde, 2% glutaraldehyde in 0.1M sodium cacodylate buffer, pH 7.4 for 30 minutes at 4° C. and processed for CMPase cytochemistry as previously described (31). Morphometric quantification of AVs was done on 20 electron micrographs per condition using Image J software T-test analysis was performed and P value (two-tailed significance) under or equal to 0.05 was considered significant (marked with asterisk).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCE

1. S. M. Noorwez et al., Journal of Biological Chemistry 279:16278-16284 (2004)

1. A method for treating or preventing a protein conformation disorder (PCD) in a subject, the method comprising administering an effective amount of a compound that enhances autophagic protein degradation to the subject.

2. (canceled)

3. The method of claim 1, wherein the compound is selected from the group consisting of: rapamycin, farnesyl transferase inhibitor, FTI-277, or an analog thereof.

4-10. (canceled)

11. The method of claim 1, wherein the method further comprises administering 11-cis-retinal, 9-cis-retinal, or a 7-ring locked isomer of 11-cis-retinal to the subject.

12. A method for treating or preventing an ocular protein conformation disorder (PCD) selected from the group consisting of retinitis pigmentosa, wet or dry age-related macular degeneration, glaucoma, corneal dystrophies, retinoschises, Stargardt's disease, autosomal dominant druzen, and Best's macular dystrophy in a subject, the method comprising administering an effective amount of rapamycin, a farnesyl transferase inhibitor, FTI-277, or an analog thereof.

13-19. (canceled)

20. The method of claim 12, wherein the method further comprises administering 11-cis-retinal, 9-cis-retinal, or a 7-ring locked isomer of 11-cis-retinal to the subject that enhances autophagic protein degradation to the subject.

14-26. (canceled)

27. The method of claim 1, wherein the subject comprises a mutation that affects protein folding.

28-29. (canceled)

30. The method of claim 1, wherein the degradation is selective for the misfolded protein.

31-34. (canceled)

35. The method of claim 12, wherein the administration is intra-ocular.

36. The method of claim 12, wherein the 11-cis-retinal or 9-cis-retinal and the compound are each incorporated into a microsphere, nanosphere, or nanoemulsion-composition or a drug delivery device that provides for their long-term release.

37-45. (canceled)

46. The method of claim 1, further comprising the step of identifying the patient as having a PCD.

47. The method of claim 1, further comprising the step of measuring the level or expression of a misfolded protein, an autophagic marker or autophagic vacuoles in a cell.

48-53. (canceled)

54. A method of enhancing the degradation of a misfolded protein in a cell, the method comprising contacting a cell with an effective amount of a compound selected from the group consisting of rapamycin, a farnesyl transferase inhibitor, FTI-277, or an analog thereof that enhances autophagy.

55-60. (canceled)

61. The method of claim 54, wherein the cell is an ocular cell.

62. The method of claim 61, wherein the method further comprises contacting the ocular cell with 11-cis-retinal, 9-cis-retinal, or a 7-ring locked isomer of 11-cis-retinal.

63-72. (canceled)

73. A pharmaceutical composition for the treatment of a PCD comprising a mammalian target of rapamycin (mTOR) inhibitor, a Ras homolog enriched in brain (Rheb) inhibitor, or a compound that enhances autophagy, or an analog thereof in a pharmaceutically acceptable excipient.

74-75. (canceled)

76. The method of claim 73, wherein the compound is rapamycin, farnesyl transferase inhibitor, FTI-277 or an analog thereof.

77-78. (canceled)

79. The method of claim 73, wherein the composition further comprises an effective amount of 11-cis-retinal or 9-cis-retinal.

80-83. (canceled)

84. A kit for the treatment of an ocular PCD, the kit comprising an effective amount of 11-cis-retinal or 9-cis-retinal and an effective amount of rapamycin or an analog thereof.

85. (canceled)

86. A method for identifying a compound useful for treating a subject having a PCD, the method comprising
a) contacting a cell in vitro expressing a misfolded protein with a candidate compound; and
b) determining an increase in autophagy in the cell relative to a control cell, wherein an increase in autophagy in the contacted cell identifies a compound useful for treating a subject having a PCD.

87. A method for identifying a compound useful for treating a subject having retinitis pigmentosa or age-related macular degeneration, the method comprising
a) contacting a cell expressing a misfolded protein in vitro with
(i) 11-cis-retinal or 9-cis-retinal, and
(ii) a candidate compound; and
b) determining an increase in autophagy in the cell relative to a control cell, wherein an increase in autophagy in the contacted cell identifies a compound useful for treating a subject having retinitis pigmentosa.

88-95. (canceled)

96. A method for treating or preventing a protein conformation disorder (PCD) in a subject, the method comprising administering an effective amount of a compound that enhances a rapamycin or FTI-277 biological activity.

97-103. (canceled)

104. A method for treating or preventing a protein conformation disorder (PCD) in a subject, the method comprising
a) administering to the subject rapamycin and a compound that enhances a rapamycin biological activity; and
b) administering 11-cis-retinal or 9-cis-retinal, wherein the 11-cis-retinal or 9-cis-retinal and the compound are administered simultaneously or within fourteen days of each other in amounts sufficient to treat or prevent retinitis pigmentosa in the subject.

105. (canceled)

106. A method of enhancing the degradation of a misfolded protein in a cell, the method comprising contacting a cell with an effective amount of rapamycin or an analog thereof and a compound that enhances a rapamycin biological activity, wherein rapamycin and the compound are each administered in an amount sufficient to enhance degradation of the protein.

107. (canceled)

108. The method of claim 106, wherein the method further comprises contacting the cell with 11-cis-retinal, 9-cis-retinal, or a 7-ring locked isomer of 11-cis-retinal.

109. A pharmaceutical composition for the treatment of an ocular PCD comprising rapamycin or an analog thereof and a compound that enhances a rapamycin biological activity in a pharmaceutically acceptable excipient, wherein rapamycin and the compound are each present in an amount sufficient to treat or prevent the PCD in the subject.

110. A method for identifying a compound useful for treating a subject having a PCD, the method comprising
a) contacting a cell in vitro expressing a misfolded protein with a candidate compound in the presence or absence of an autophagy enhancer; and
b) determining an increase in autophagy in the cell relative to a control cell, wherein an increase in autophagy in the contacted cell identifies a compound useful for treating a subject having a PCD.

111. A method for identifying a compound useful for treating a subject having retinitis pigmentosa, the method comprising
a) contacting a cell expressing a misfolded opsin protein in vitro with
(i) 11-cis-retinal or 9-cis-retinal and rapamycin, and
(ii) a candidate compound; and
b) determining an increase in autophagy in the cell relative to a control cell, wherein an increase in autophagy in the contacted cell identifies a compound useful for treating a subject having retinitis pigmentosa.

112. (canceled)
